(19) United States
(12) Patent Application Publication
(10) Pub. No.: US 2021/0369734 A1
(43) Pub. Date: Dec. 2, 2021

(54) COMPOUNDS AND METHODS OF THEIR USE

(71) Applicant: Sage Therapeutics, Inc., Cambridge, MA (US)

(72) Inventor: James J. Doherty, Bedford, MA (US)

(21) Appl. No.: 17/401,787

(22) Filed: Aug. 13, 2021

Related U.S. Application Data
(63) Continuation of application No. 15/777,394, filed on May 18, 2018.
(60) Provisional application No. 62/258,222, filed on Nov. 20, 2015.

(51) Int. Cl. A61K 31/569; A61P 25/28; A61P 25/22; A61P 25/08; A61K 9/00; A61K 31/568; A61K 31/5685; A61K 31/573; A61K 31/58

(52) U.S. Cl. CPC A61K 31/569; A61P 25/28; A61P 25/22; A61P 25/08; A61K 31/58; A61K 31/568; A61K 31/5685; A61K 31/573; A61K 9/0019

(57) ABSTRACT

Compounds, compositions, and methods for treating injuries caused by exposure to chemical warfare and similar agents are described herein.

### CLAIM OF PRIORITY

This application claims priority from U.S. Ser. No. 62/258,222 filed Nov. 20, 2015, which is incorporated herein by reference in its entirety.

### BACKGROUND

Brain excitability is defined as the level of arousal of an animal, a continuum that ranges from coma to convulsions, and is regulated by various neurotransmitters. In general, neurotransmitters are responsible for regulating the conductance of ions across neuronal membranes. At rest, the neuronal membrane possesses a potential (or membrane voltage) of approximately −70 mV, the cell interior being negative with respect to the cell exterior. The potential (voltage) is the result of ion (K⁺, Na⁺, Cl⁻, organic anions) balance across the neuronal semipermeable membrane. Neurotransmitters are stored in presynaptic vesicles and are released under the influence of neuronal action potentials. When released into the synaptic cleft, an excitatory chemical transmitter such as acetylcholine will cause membrane depolarization, e.g., a change of potential from −70 mV to −50 mV. This effect is mediated by postsynaptic nicotinic receptors which are stimulated by acetylcholine to increase membrane permeability to Na⁺ ions. The reduced membrane potential stimulates neuronal excitability in the form of a postsynaptic action potential.

In the case of the GABA receptor complex (GRC), the effect on brain excitability is mediated by GABA, a neurotransmitter. GABA has a profound influence on overall brain excitability because up to 40% of the neurons in the brain utilize GABA as a neurotransmitter. GABA regulates the excitability of individual neurons by regulating the conductance of chloride ions across the neuronal membrane. GABA interacts with its recognition site on the GRC to facilitate the flow of chloride ions down an electrochemical gradient of the GRC into the cell. An intracellular increase in the levels of this anion causes hyperpolarization of the transmembrane potential, rendering the neuron less susceptible to excitatory inputs, i.e., reduced neuron excitability. In other words, the higher the chloride ion concentration in the neuron, the lower the brain excitability and level of arousal.

It is well-documented that the GRC is responsible for the mediation of anxiety, seizure activity, and sedation. Thus, GABA and drugs that act like GABA or facilitate the effects of GABA (e.g., the therapeutically useful barbiturates and benzodiazepines (BZs), such as Valium®) produce their therapeutically useful effects by interacting with specific regulatory sites on the GRC. Accumulated evidence has now indicated that in addition to the benzodiazepine and barbiturate binding site, the GRC contains at least one distinct site for interaction with neuroactive steroids. See, e.g., Lan, N. C. et al., Neurochem. Res. (1991) 16:347-356.

Neuroactive steroids can occur endogenously. The most potent endogenous neuroactive steroids are 3α-hydroxy-5-reduced pregnan-20-one and 3α-21-dihydroxy-5-reduced pregnan-20-one, metabolites of hormonal steroids progesterone and deoxycorticosterone, respectively. The ability of these steroid metabolites to alter brain excitability was recognized in 1986 (Majewska, M. D. et al., Science 232:1004-1007 (1986); Harrison, N. L. et al., J Pharmacol. Exp. Ther. 241:346-353 (1987)).

New and improved neuroactive steroids are needed that act as modulating agents for brain excitability, as well as agents for the prevention and treatment of CNS-related diseases. The compounds, compositions, and methods described herein are directed toward this end.

### SUMMARY OF THE INVENTION

Described herein are compounds and their methods of use for treating subjects (e.g., subjects injured by chemical warfare and similar agents). For example, the compounds and methods described herein treat or prevent injury resulting from a chemical warfare or similar agent. In some embodiments, the injury is a seizure (e.g., seizure activity for greater than five minutes). In some embodiments, the injury is status epilepticus.

In some embodiments, the compound is a neuroactive steroid. In some embodiments, the compound (e.g., the neuroactive steroid) is pregnanolone, allopregnanolone, alphadalone, ganaxolone, or alphaxolone. In some embodiments, the neuroactive steroid is pregnanolone, allopregnanolone, alphadalone, ganaxolone, or alphaxolone. In some embodiments, the compound (e.g., the neuroactive steroid) is allopregnanolone. In some embodiments, the compound (e.g., the neuroactive steroid) is selected from neuroactive steroids that are disclosed in WIPO Publication Nos. WO2013/188792, WO 2013/056181, WO2015/010054, WO2014/169832, WO2014/169836, WO2014/169833, WO2014/169831, WO2015/027227, WO 2014/100228; U.S. Publication No. US20140275241, US20140256805; U.S. Pat. Nos. 5,232,917, 8,575,375 and 8,759,330.

In an aspect, provided herein is a method for treating a subject having an injury resulting from exposure to a warfare agent (e.g., a chemical warfare agent), the method comprising administering to the subject a compound described herein (e.g., a GABA modulator such as a compound (e.g., neuroactive steroid) described herein).

In an aspect, provided herein is a method of treating an injury in a subject who has been exposed to a chemical warfare agent, the method comprising administering to the subject a compound described herein (e.g., GABA modulator such as a compound (e.g., neuroactive steroid) described herein).

In an aspect, provided herein is a method of treating a subject, the method comprising: identifying a subject that has been exposed to a chemical warfare agent such as a nerve agent or toxin; and administering to the subject a compound described herein (e.g., GABA modulator such as a neuroactive steroid described herein).

In some embodiments, the injury is a seizure. In some embodiments, the injury is a myoclonic seizure (e.g., sporadic jerks).

In some embodiments, the injury is status epilepticus.

In some embodiments, the administration is within 1 week; 6, 5, 4, 3, 2, 1 day; 24, 22, 20, 18, 16, 14, 12, 10, 8, 7, 6, 5, 4, 3, 2, 1 hour, 45, 30, 20, 10, or 5 minutes of exposure to the chemical warfare agent.

In some embodiments, the compound is administered parenterally. In some embodiments, the compound is administered by intravenous administration.

In some embodiments, the subject is a human.

In some embodiments, the chemical warfare agent is a nerve agent or toxin. In some embodiments, the chemical warfare agent is a nerve agent. In some embodiments, the nerve agent is a phosphorus-containing organic chemical. In some embodiments, the nerve agent is a G agent (e.g., tabun (GA), sarin (GB), soman (GD), cyclosarin (GF), and GV). In some embodiments, the nerve agent is a V agent (e.g., VE, VG, VM, VX, and Novichok agents). In some embodiments, the toxin is abrin, ricin, or saxitoxin.

In some embodiments, the compound is a compound as described herein.

In an aspect, provided herein is a method for treating disorders related to GABA function in a subject in need thereof, the method comprising administering to the subject a therapeutically effective amount of a compound, a pharmaceutically acceptable salt thereof, or pharmaceutical composition of a compound described herein.

In an aspect, provided herein is a method for treating a CNS-related disorder in a subject in need thereof, comprising administering to the subject an effective amount of a compound described herein, or a pharmaceutically acceptable salt thereof.

In some embodiments, the CNS-related disorder is a sleep disorder, a mood disorder such as depression, a schizophrenia spectrum disorder, a convulsive disorder, epileptogenesis, a disorder of memory and/or cognition, a movement disorder, a personality disorder, autism spectrum disorder, pain, traumatic brain injury, a vascular disease, a substance abuse disorder and/or withdrawal syndrome, or tinnitus.

In an aspect, provided herein is a kit comprising a solid composition comprising a compound as described herein and a sterile diluent.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 8 carbon atoms (“C₁₋₈ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(—CH₃)₂), nPr (—CH₂CH₂CH₃), n-Bu (—CH₂CH₂CH₂CH₃), or i-Bu (—CH₂CH(CH₃)₂).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 8 carbon atoms (“C₂₋₈ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 8 carbon atoms (“C₂₋₈ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“Cₘ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Aryl groups include, but are not limited to, phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

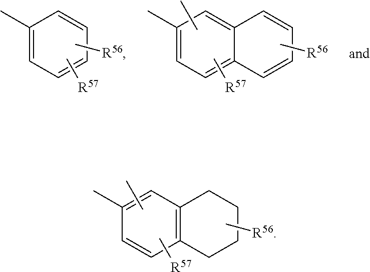

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹ NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

Other representative aryl groups having a fused heterocyclyl group include the following:

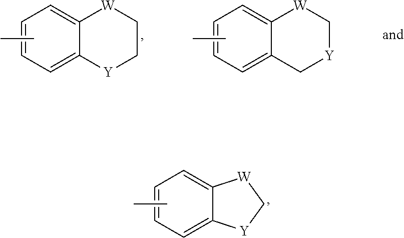

wherein each W is selected from C(R⁶⁶)₂, NR⁶⁶, O, and S; and each Y is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

“Haloalkyl” and “haloalkoxy” can include alkyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Hydroxy” or “hydroxyl,” independently or as part of another substituent, mean, unless otherwise stated, a —OH group.

Hydroxyalkyl” or “hydroxylalkyl” can include alkyl structures that are substituted with one or more hydroxyl groups.

“Heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following formulae:

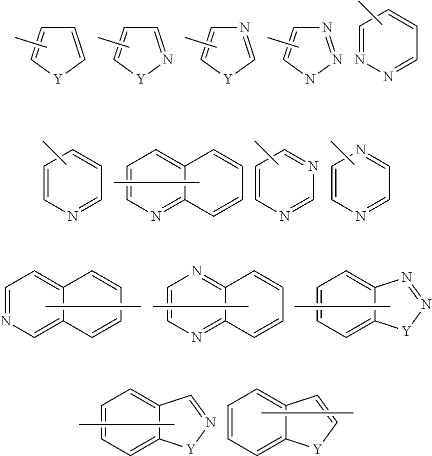

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Carbocyclyl” or “carbocyclic” refers to a radical of a nonaromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered nonaromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered nonaromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

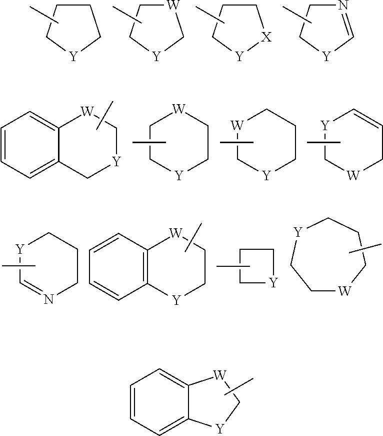

wherein each W is selected from CR⁶⁷, C(R⁶⁷)₂, NR⁶⁷, O, and S; and each Y is selected from NR⁶⁷, O, and S; and R⁶⁷ is independently hydrogen, C₁-C₈ alkyl, C₃-C₁₀ cycloalkyl, 4-10 membered heterocyclyl, C₆-C₁₀ aryl, and 5-10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (e.g., amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Acyl” refers to a radical —C(O)R²⁰, where R²⁰ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁—C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Acylamino” refers to a radical —NR²²C(O)R²³, where each instance of R²² and R²³ is independently hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁-C₈ alkyl, —NR²⁴C(O)—(CH₂)ₜ(C₆-C₁₀ aryl), —NR²⁴C(O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocyclyl), wherein t is an integer from 0 to 4, and each R²⁴ independently represents hydrogen or C₁-C₈ alkyl. In certain embodiments, R²⁵ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and R²⁶ is H, C₁-C₈ alkyl, substituted with halo or hydroxy; C₃-C₁₀ cycloalkyl, 4-10-membered heterocyclyl, C₆-C₁₀ aryl, arylalkyl, 5-10-membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided at least one of R²⁵ and R²⁶ is other than H.

“Acyloxy” refers to a radical —OC(O)R²⁷, where R²⁷ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. Representative examples include, but are not limited to, formyl, acetyl, cyclohexylcarbonyl, cyclohexylmethylcarbonyl, benzoyl, and benzylcarbonyl. In certain embodiments, R²⁸ is C₁-C₈ alkyl, substituted with halo or hydroxy; C₃-C₁₀ cycloalkyl, 4-10-membered heterocyclyl, C₆-C₁₀ aryl, arylalkyl, 5-10-membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxy, nitro, thioalkoxy, thioaryloxy, thiol, alkyl-S(O)—, aryl-S(O)—, alkyl-S(O)₂— and aryl-S(O)₂—. Exemplary “substituted alkoxy” groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents hydrogen or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R⁶² is not a hydrogen. In certain embodiments, R⁶² is selected from H, C₁-C₈ alkyl, C₃-C₁₀ cycloalkyl, 4-10 membered heterocyclyl, C₆-C₁₀ aryl, and 5-10 membered heteroaryl; or C₁-C₈ alkyl substituted with halo or hydroxy; or C₃-C₁₀ cycloalkyl, 4-10 membered heterocyclyl, C₆-C₁₀ aryl, or 5-10 membered heteroaryl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁶² is other than H.

“Carboxy” refers to the radical —C(O)OH.

“Cyano” refers to the radical —CN.

“Nitro” refers to the radical —NO₂.

“Ethenyl” refers to substituted or unsubstituted —(C═C)—. “Ethylene” refers to substituted or unsubstituted —(C—C)—. “Ethynyl” refers to —(C≡C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)S R^{aa}, —SC(═S)S R^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR')R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂,—NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl),-OC(═NH)(C₁₋₆ alkyl), —OC(═NH)OC₁₋₆ alkyl, —C(═NH)N(C₁₋₆ alkyl), —C(═NH)NH(C₁₋₆ alkyl), —C(═NH)NH₂, —OC(═NH)N(C_{1 6} alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂,—SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ -C(═S)N(C₁₋₆ alkyl), C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl), —OP(═O)(C₁₋₆ alkyl)₂, —OP(═O)(OC_{1 6} alkyl)₂, C₁₋₆ alkyl, C_{1 6} perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cr, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —S OR^{aa}, —C(═S)N(R^{cc})₂, —C(═O)S R^{cc}, —C(═S)S R^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14-membered heteroaryl, or two 12′ groups attached to a nitrogen atom are joined to form a 3-14-membered heterocyclyl or 5-14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is an amino protecting group (also referred to herein as a nitrogen protecting group). Amino protecting groups include, but are not limited to, —OH, —OR^{aa}, —N(R^{cc})₂, —C(═O)R^{aa}, —C(═O)OR^{aa}, —C(═O)N(R^{cc})₂, —S(═O)₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —S OR^{aa}, —C(═S)N(R^{cc})₂, —C(═O)SR^{cc}, —C(═S)SR^{cc}, C₁₋₁₀ alkyl, C₂₋₁₀ alkenyl, C₂₋₁₀ alkynyl, C₃₋₁₀ carbocyclyl, 3-14-membered heterocyclyl, C₆₋₁₄ aryl, and 5-14-membered heteroaryl groups, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary amino protecting groups include, but are not limited to amide groups (e.g., —C(═O)R^{aa}), which include, but are not limited to, formamide and acetamide; carbamate groups (e.g., —C(═O)OR^{aa}), which include, but are not limited to, 9-fluorenylmethyl carbamate (Fmoc), t-butyl carbamate (BOC), and benzyl carbamate (Cbz); sulfonamide groups (e.g., —S(═O)₂R^{aa}), which include, but are not limited to, p-toluenesulfonamide (Ts), methanesulfonamide (Ms), and N-[2-(trimethylsilyl)ethoxy]methylamine (SEM).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —

P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), 2-methoxyethoxymethyl (MEM), benzyl (Bn), triisopropylsilyl (TIPS), t-butyldimethylsilyl (TBDMS), t-butylmethoxyphenylsilyl (TBMPS), methanesulfonate (mesylate), and tosylate (Ts).

In certain embodiments, the substituent present on an sulfur atom is an sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and Claims. The invention is not intended to be limited in any manner by the above exemplary listing of substituents.

### Other Definitions

As used herein, the term “modulation” refers to the inhibition or potentiation of GABA receptor function. A “modulator” (e.g., a modulator compound) may be, for example, an agonist, partial agonist, antagonist, or partial antagonist of the GABA receptor.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are nontoxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g. in crystalline form and may be solvated or hydrated. Suitable solvates include pharmaceutically acceptable solvates, such as hydrates, and further include both stoichiometric solvates and nonstoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O, and ¹³_{N}, and would be useful in Positron Emission Topography (PET) studies for examining substrate receptor occupancy. All isotopic variants of the compounds provided herein, radioactive or not, are intended to be encompassed within the scope of the invention.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, humans (i.e., a male or female of any age group, e.g., a pediatric subject (e.g, infant, child, adolescent) or adult subject (e.g., young adult, middleaged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### DETAILED DESCRIPTION OF CERTAIN EMBODIMENTS OF THE INVENTION

Compounds and methods of their use for treating subjects (e.g., subjects injured by chemical warfare and similar agents) are described herein.

### Chemical Warfare Agents

A subject may be exposed to a chemical warfare agent. If a compound described herein is administered, the symptoms or injuries resulting from the exposure to the chemical warfare agents can be reduced, prevented, or both. The compounds described herein can be administered to a subject before, during, or following such exposure and is therefore administered within 1 week; 6, 5, 4, 3, 2, 1 day; 24, 22, 20, 18, 16, 14, 12, 10, 8, 7, 6, 5, 4, 3, 2, 1 hour, 45, 30, 20, 10, or 5 minutes before or after such exposure. The compounds described herein can be administered prophylactically, when exposure to an agent is anticipated. It can also be administered after exposure to the chemical warfare agent (e.g., before or after symptoms of injury present in a subject).

Injuries resulting from the exposure to chemical warfare agents are known in the art and include any physical injuries, such as injuries to the central nervous system and peripheral nervous system. Exemplary symptoms or injuries resulting from the exposure to chemical warfare agents include inflammation, burn, itch, pain, rash, blisters, sweating, muscle twitching, nausea, vomiting, diarrhea, weakness, loss of conciousness, convulsions, muscular twitching, paralysis, secretions (from the mouth, nose, or lung for example), difficulty breating, blurred vision, eye pain, lacrimation, red eyes, shortness of breath, coughing, phlegm production and narrowing of the airways, headaches, tremors, dizziness, numbness or tingling, anxiety, insomnia, depression, emotional instability, and even death. The term “chemical warfare agent” includes all of those agents classified as schedule 1, 2, and 3 agents under the Chemical Weapons Convention of 1993 and may be in liquid form, gas form, solid form, or combinations thereof. Exemplary agents are described in further detail below and include, for example, nerve agents and toxins.

Nerve agents. Nerve agent poisoning typically leads to contraction of pupils, profuse salivation, convulsions, involuntary urination and defecation, and eventual death by asphyxiation as control is lost over respiratory muscles. For example, nerve agents can be phosphorus-containing organic chemicals (organophosphates) that disrupt the mechanism by which nerves transfer messages to organs. Exemplary agents include G agents such as tabun (GA), sarin (GB), soman (GD), cyclosarin (GF), and GV and V agents such as VE, VG, VM, VX, and Novichok agents.

Toxins. Exemplary toxins are abrin, ricin, and saxitoxin.

### Compounds

Provided herein are compounds and their methods of use for treating subjects (e.g., subjects injured) by chemical warfare and similar agents.

Various synthetic steroids have also been prepared as neuroactive steroids. See, for example, U.S. Pat. No. 5,232,917, which discloses neuroactive steroid compounds useful in treating stress, anxiety, insomnia, seizure disorders, and mood disorders such as depression, that are amenable to GRC-active agents, such as depression, in a therapeutically beneficial manner. Furthermore, it has been previously demonstrated that these steroids interact at a site or sites on the GRC which is distinct from other known sites of interaction (e.g., barbiturates, benzodiazepines, and GABA) where therapeutically beneficial effects on stress, anxiety, sleep, mood disorders and seizure disorders have been previously elicited (see, e.g., Gee, K. W. and Yamamura, H. I., “Benzodiazepines and Barbiturates: Drugs for the Treatment of Anxiety, Insomnia and Seizure Disorders,” in Central Nervous System Disorders, Horvell, ed., Marcel-Dekker, New York (1985), pp. 123-147; Lloyd, K. G. and Morselli, P. L., “Psychopharmacology of GABAergic Drugs,” in Psychopharmacology: The Third Generation of Progress, H. Y. Meltzer, ed., Raven Press, N.Y. (1987), pp. 183-195; and Gee et al., European Journal of Pharmacology, 136:419-423 (1987). These compounds are desirable for their duration, potency, and oral activity (along with other forms of administration).

Compounds described herein are generally designed to modulate GABA function, and therefore to act as neuroactive steroids for the treatment and prevention of CNS-related conditions in a subject. Modulation, as used herein, refers to the inhibition or potentiation of GABA receptor function. Accordingly, the compounds and pharmaceutical compositions provided herein find use as therapeutics for preventing and/or treating CNS conditions in mammals including humans and non-human mammals. Thus, and as stated earlier, the present invention includes within its scope, and extends to, the recited methods of treatment, as well as to the compounds for such methods, and to the use of such compounds for the preparation of medicaments useful for such methods.

In some embodiments, the compounds described herein (e.g., neuroactive steroid described herein) is pregnanolone, allopregnanolone, alphadalone, ganaxolone, or alphaxolone. In some embodiments, the compound (e.g., the neuroactive steroid) is pregnanolone, allopregnanolone, alphadalone, ganaxolone, or alphaxolone. In some embodiments, the neuroactive steroid is allopregnanolone. In some embodiments, the neuroactive steroid is selected from neuroactive steroids that are disclosed in WIPO Publication Nos. WO2013/188792, WO 2013/056181, WO2015/010054, WO2014/169832, WO2014/169836, WO2014/169833, WO2014/169831, WO2015/027227, WO 2014/100228, U.S. Pat. Nos. 5,232,917, 8,575,375 and 8,759,330.

In one aspect, provided is a compound of Formula (I):

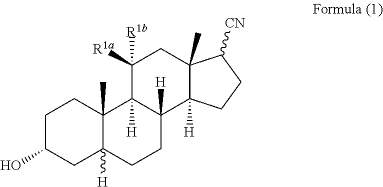

wherein R^{1a} is hydrogen, halo, alkyl, alkoxy, —C(O)R^{a}, —C(O)N(R^{b})(R^{c}), —C(O)OR^{a}, —N(R^{b})(R^{c}), —OC(O)N(R^{b})(R^{c}), —OC(O)OR^{a}, —S(O)₀₋₂R^{a}, —S(O)₀₋₂OR^{a}, or —S(O)₀₋₂N(R^{b})(R^{c}); R^{1b} is hydrogen, halo, hydroxy, alkyl, alkoxy, —C(O)R^{a}, —C(O)N(R^{b})(R^{c}), —C(O)OR^{a}, —N(R^{d})(R^{e}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); wherein one of R^{1a} and R^{1b} is hydrogen; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form a 3-7-membered (e.g., 5-7-membered) heterocyclic ring; and each R^{d} and R^{e} is hydrogen, substituted methyl or C₂-C₆ alkyl, or R^{d} and R^{e}, taken together with the nitrogen atom to which they are attached, form a 3-7-membered (e.g., 5-7-membered) heterocyclic ring.

In some embodiments, R^{1a} is hydrogen and R^{1b} is hydroxy, alkyl, or alkoxy. In some embodiments, R^{1b} is hydroxy or alkoxy. In some embodiments, R^{1b} is hydroxy. In some embodiments, R^{1b} is methoxy. In some embodiments, R^{1b} is alkyl.

In some embodiments, R^{1b} is hydrogen and R^{1a} is alkyl or alkoxy. In some embodiments, R^{1a} is alkoxy. In some embodiments, R^{1a} is methoxy.

In some embodiments, the compound is of the Formula (Ia):

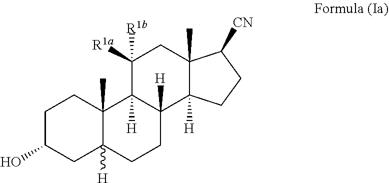

wherein R^{1a} is hydrogen, halo, alkyl, alkoxy, —C(O)R^{a}, —C(O)N(R^{b})(R^{c}), —C(O)OR^{a}, —N(R^{b})(R^{c}), —OC(O)N(R^{b})(R^{c}), —OC(O)OR^{a}, —S(O)₀₋₂R^{a}, —S(O)₀₋₂OR^{a}, or —S(O)₀₋₂N(R^{b})(R^{c}); R^{1b} is hydrogen, halo, hydroxy, alkyl, alkoxy, —C(O)R^{a}, C(O)N(R^{b})(R^{c}), —C(O)OR^{a}, —N(R^{d})(R^{e}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); wherein one of R^{1a} and R^{1b} is hydrogen; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form a 3-7-membered (e.g., 5-7-membered) heterocyclic ring; and each R^{d} and R^{e} is hydrogen, substituted methyl or C₂-C₆ alkyl, or R^{d} and R^{e}, taken together with the nitrogen atom to which they are attached, form a 3-7-membered (e.g., 5-7-membered) heterocyclic ring.

In some embodiments, R^{1a} is hydrogen and R^{1b} is hydroxy, alkyl, or alkoxy. In some embodiments, R^{1b} is hydroxy or alkoxy. In some embodiments, R^{1b} is hydroxy. In some embodiments, R^{1b} is methoxy. In some embodiments, R^{1b} is alkyl.

In some embodiments, R^{1b} is hydrogen and R^{1a} is alkyl or alkoxy. In some embodiments, R^{1a} is alkoxy. In some embodiments, R^{1a} is methoxy.

In some embodiments, the compound is of the Formula (Ib):

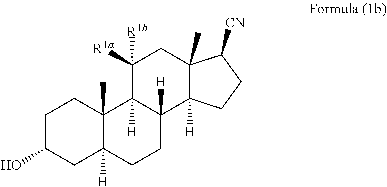

wherein R^{1a} is hydrogen, halo, alkyl, alkoxy, —C(O)R^{a}, —C(O)N(R^{b})(R^{c}), —C(O)OR^{a}, —N(R^{b})(R^{c}), —OC(O)N(R^{b})(R^{c}), —OC(O)OR^{a}, —S(O)₀₋₂R^{a}, —S(O)₀₋₂OR^{a}, or —S(O)₀₋₂N(R^{b})(R^{c}); R^{1b} is hydrogen, halo, hydroxy, alkyl, alkoxy, —C(O)R^{a}, —C(O)N(R^{b})(R^{c}), —C(O)OR^{a}, —N(R^{d})(R^{e}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); wherein one of R^{1a} and R^{1b} is hydrogen; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form a 3-7-membered (e.g., 5-7-membered) heterocyclic ring; and each R^{d} and R^{e} is hydrogen, substituted methyl or C₂-C₆ alkyl, or R^{d} and R^{e}, taken together with the nitrogen atom to which they are attached, form a 3-7-membered (e.g., 5-7-membered) heterocyclic ring.

In some embodiments, R^{1a} is hydrogen and R^{1b} is hydroxy, alkyl, or alkoxy. In some embodiments, R^{1b} is hydroxy or alkoxy. In some embodiments, R^{1b} is hydroxy. In some embodiments, R^{1b} is methoxy. In some embodiments, R^{1b} is alkyl.

In some embodiments, R^{1b} is hydrogen and R^{1a} is alkyl or alkoxy. In some embodiments, R^{1a} is alkoxy. In some embodiments, R^{1a} is methoxy.

In one aspect, provided is a compound selected from:

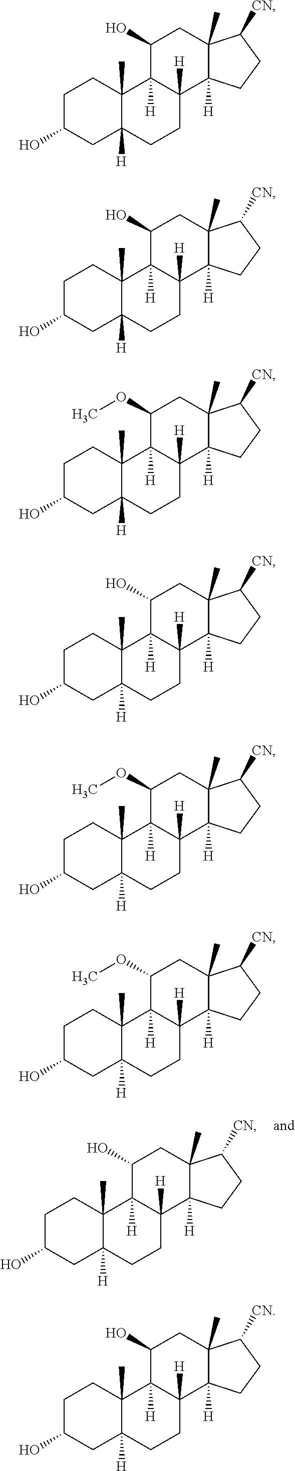

In some embodiments, the compound is selected from:

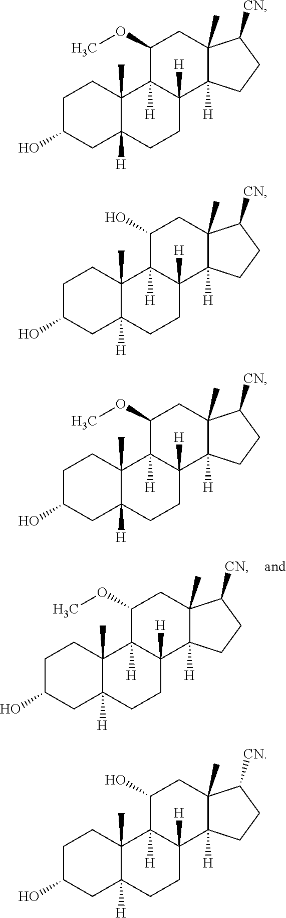

In some embodiments, the compound is selected from:

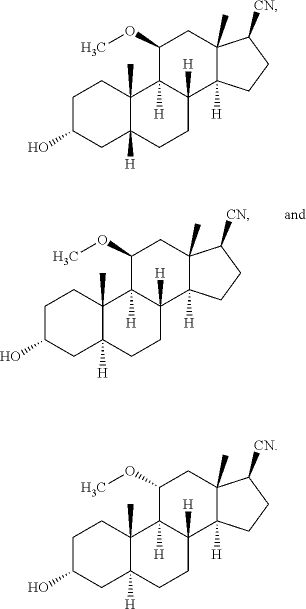

In one aspect, provided is a compound of the Formula (II):

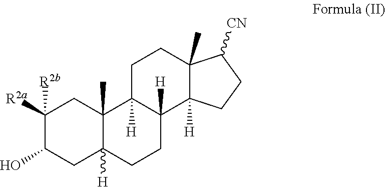

wherein R^{2a} is hydrogen, halo (e.g., chloro. fluoro. bromo. iodo). hydroxy, alkyl, methoxy, substituted ethoxy, C₃-C₆ alkoxy, —C(O)R^{a}, —C(O)N(R^{b})(R^{c}), —C(O)OR^{a}, —N(R^{f})(R^{g}), —OC(O)N(R^{b})(R^{c}), —OC(O)OR^{a}, —OC(O)R^{a}, —S(O)₀₋₂R^{a}, −S(O)₀₋₂OR^{a}, or —S(O)₀₋₂N(R^{b})(R^{c}); R^{2b} is hydrogen, halo, hydroxy, alkyl, methoxy. substituted ethoxy. C₃-C₆ alkoxy, —C(O)R^{a}, —C(O)N(R^{b})(R^{c}), —C(O)OR^{a}, —N(R^{b})(R^{c}). —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); wherein one of R^{2a} and R^{2b} is hydrogen; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form a 3-7-membered (e.g., 5-7-membered) heterocyclic ring; and each R^{f} and R^{g} is independently hydrogen or C₁-C₆ alkyl, or R^{f} and R^{g}, taken together with the nitrogen atom to which they are attached, form a 3-7-membered (e.g., 5-7-membered) heterocyclic ring optionally comprising one additional heteroatom selected from nitrogen, oxygen and sulfur.

In some embodiments, R^{2a} is hydrogen and R^{2b} is alkyl. In some embodiments, R^{2b} is alkyl. In some embodiments, R^{2b} is methyl. In some embodiments, R^{2b} is methoxy.

In some embodiments, R^{2b} is hydrogen and R^{2a} is alkyl, methoxy. substituted ethoxy, or C₃-C₆ alkoxy. In some embodiments, R^{2b} is alkyl. In some embodiments, R^{2a} is methyl. In some embodiments, R^{2a} is ethyl. In some embodiments, R^{2a} is methoxy. In some embodiments, R^{2a} is —OCF₃. In some embodiments, R^{2a} is substituted ethoxy. In some embodiments, R^{2a} is —OCH₂CH₂OMe. In some embodiments, R^{2a} is —OCH₂CH₂OH. In some embodiments, R^{2a} is —OCH₂CF₃. In some embodiments, R^{2a} is C₃-C₆ alkoxy. In some embodiments, R^{2a} is propoxy. In some embodiments, R^{2a} is —OCH(CH₃)₂. In some embodiments, R^{2a} is —OCH₂CH(CH₃)₂. In some embodiments, R^{2a} is cyclopropoxy.

In some embodiments, the compound is of the Formula (IIa):

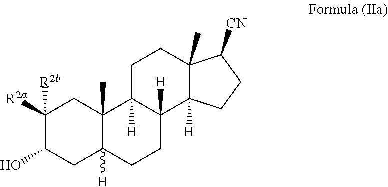

wherein R^{2a} is hydrogen, halo (e.g., chloro. fluoro. bromo. iodo). hydroxy, alkyl, methoxy. substituted ethoxy, C₃-C₆ alkoxy, —C(O)R^{a}, —C(O)N(R^{b})(R^{c}), —C(O)OR^{a}, —N(R^{f})(R^{g}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); R^{2b} is hydrogen, halo, hydroxy, alkyl, methoxy, substituted ethoxy. C₃-C₆ alkoxy, —C(O)R^{a}, —C(O)N(R^{b})(R^{c}), —C(O)OR^{a}, —N(R^{b})(R^{c}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); wherein one of R^{2a} and R^{2b} is hydrogen; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form a 3-7-membered (e.g., 5-7-membered) heterocyclic ring; and each R1 and R^{g} is independently hydrogen or C₁-C₆ alkyl, or R^{f} and R^{g}, taken together with the nitrogen atom to which they are attached, form a 3-7-membered (e.g., 5-7-membered) heterocyclic ring optionally comprising one additional heteroatom selected from nitrogen, oxygen and sulfur.

In some embodiments, R^{2a} is hydrogen and R^{2b} is alkyl. In some embodiments, R^{2b} is alkyl. In some embodiments, R^{2b} is methyl. In some embodiments, R^{2b} is methoxy.

In some embodiments, R^{2b} is hydrogen and R^{2a} is alkyl, methoxy, substituted ethoxy, or C₃-C₆ alkoxy. In some embodiments, R^{2a} is alkyl. In some embodiments, R^{2a} is methyl. In some embodiments. R^{2a} is ethyl. In some embodiments, R^{2a} is methoxy. In some embodiments, R^{2a} is —OCF₃. In some embodiments, R^{2a} is substituted ethoxy. In some embodiments, R^{2a} is —OCH₂CH₂OMe. In some embodiments, R^{2a} is —OCH₂CH₂OH. In some embodiments, R^{2a} is —OCH₂CF₃. C₃-C₆ alkoxy. In some embodiments, R^{2a} is propoxy. In some embodiments, R^{2a} is —OCH(CH₃)₂. In some embodiments, R^{2a} is —OCH₂CH(CH₃)₂. In some embodiments, R^{2a} is cyclopropoxy.

In some embodiments, the compound is of the Formula (IIb):

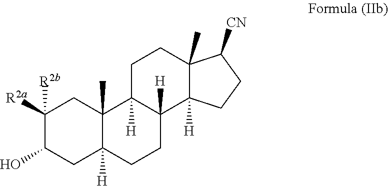

wherein R^{2a} is hydrogen, halo (e.g., chloro, fluoro, bromo, iodo), hydroxy, alkyl, methoxy, substituted ethoxy, C₃-C₆ alkoxy, —C(O)R^{a}, —C(O)N(R^{b})(R^{c}), —C(O)OR^{a}, —N(R^{f})(R^{g}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(Rb)(R^{c}); —R^{2b} is hydrogen, halo, hydroxy, alkyl, methoxy, substituted ethoxy, C₃-C₆ alkoxy, —C(O)R^{a}, —C(O)N(R^{b})(R^{c}), —C(O)OR^{a}, —N(R^{b})(R^{c}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}); wherein one of R^{2a} and R^{2b} is hydrogen; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form a 3-7-membered (e.g., 5-7-membered) heterocyclic ring; and each R^{f} and R^{g} is independently hydrogen or C₁-C₆ alkyl, or R^{f} and R^{g}, taken together with the nitrogen atom to which they are attached, form a 3-7-membered (e.g., 5-7-membered) heterocyclic ring optionally comprising one additional heteroatom selected from nitrogen, oxygen and sulfur.

In some embodiments, R^{2a} is hydrogen and R^{2b} is alkyl. In some embodiments, R^{2b} is alkyl. In some embodiments, R^{2b} is methyl. In some embodiments, R^{2b} is methoxy.

In some embodiments, R^{2b} is hydrogen and R^{2a} is alkyl, methoxy, substituted ethoxy, or C₃-C₆ alkoxy. In some embodiments, R^{2a} is alkyl. In some embodiments, R^{2a} is methyl. In some embodiments, R^{2a} is ethyl. In some embodiments, R^{2a} is methoxy. In some embodiments, R^{2a} is —OCF₃. In some embodiments, R^{2a} is substituted ethoxy. In some embodiments, R^{2a} is —OCH₂CH₂OMe. In some embodiments, R^{2a} is —OCH₂CH₂OH. In some embodiments, R^{2a} is —OCH₂CF₃. C₃-C₆ alkoxy. In some embodiments, R^{2a} is propoxy. In some embodiments, R^{2a} is —OCH(CH₃)₂. In some embodiments, R^{2a} is —OCH₂CH(CH₃)₂. In some embodiments, R^{2a} is cyclopropoxy.

In one aspect, provided is a compound selected from:

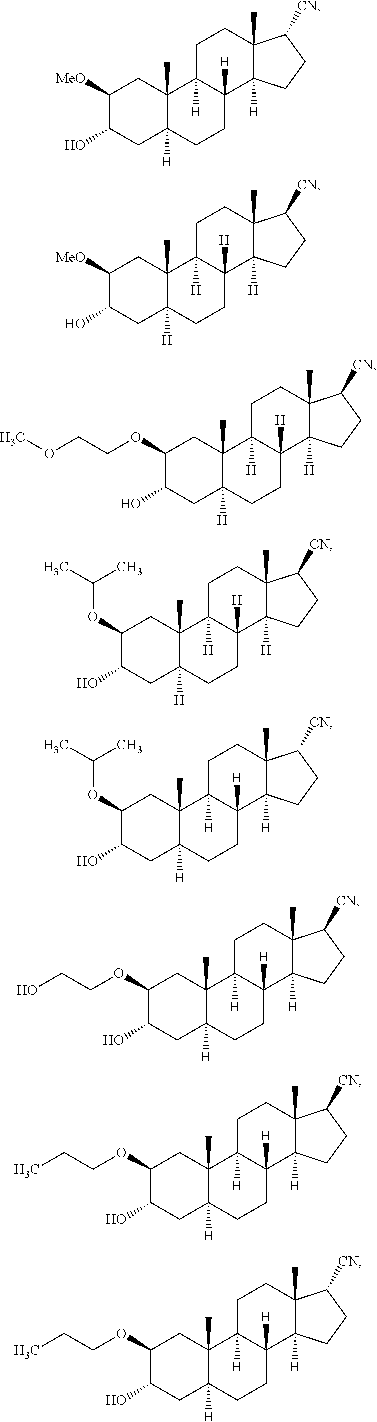

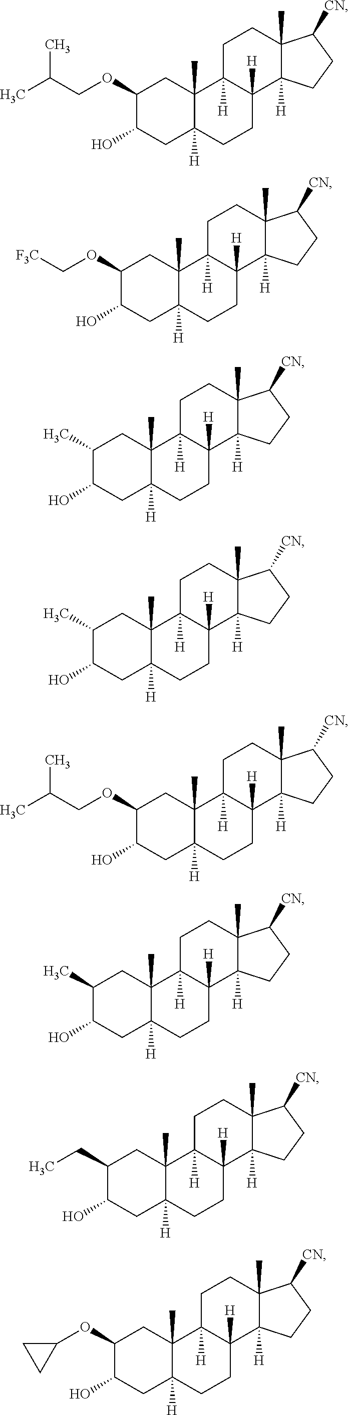

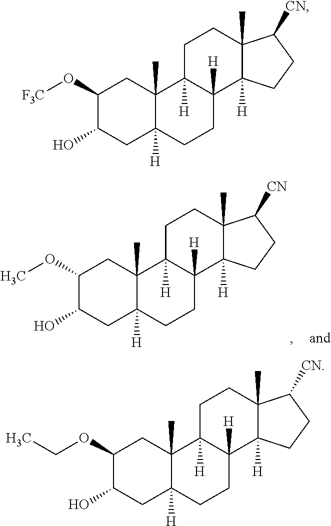

In some embodiments, the compound is selected from:

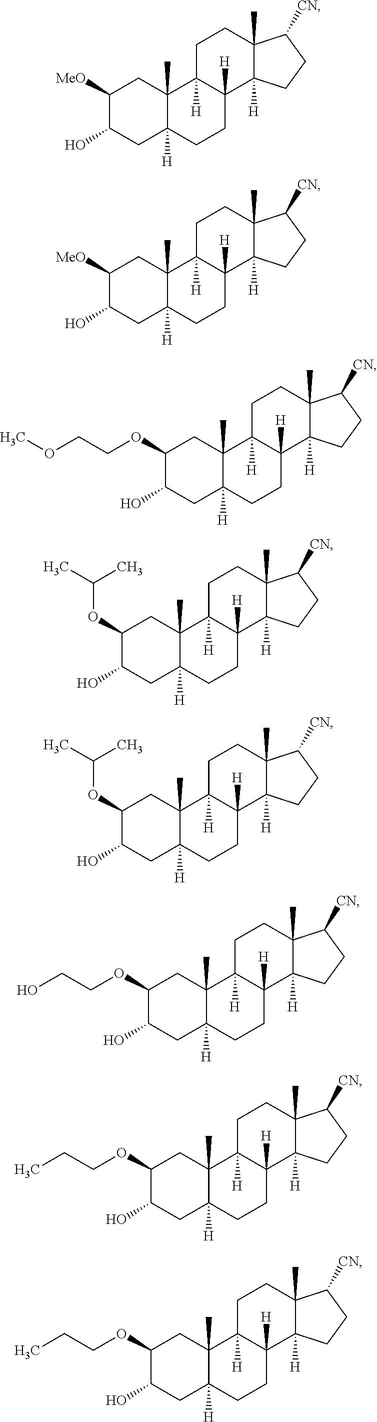

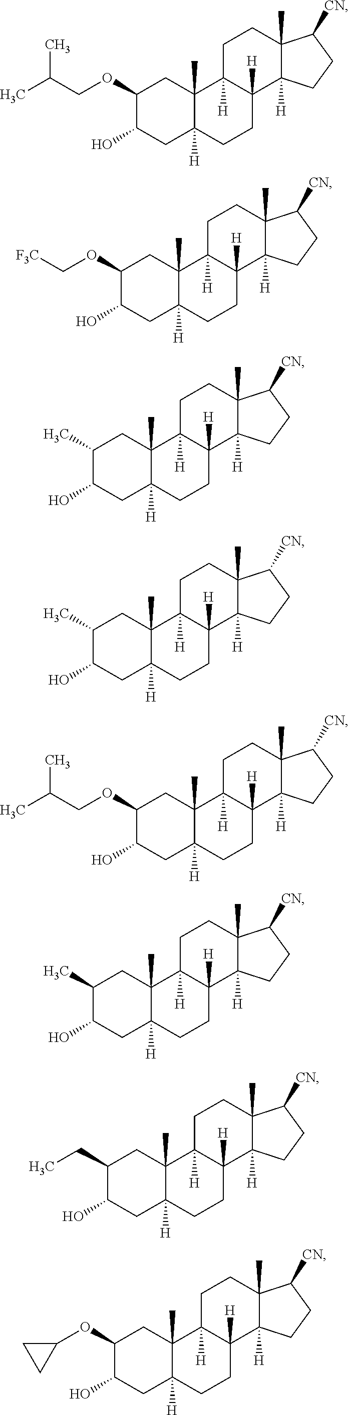

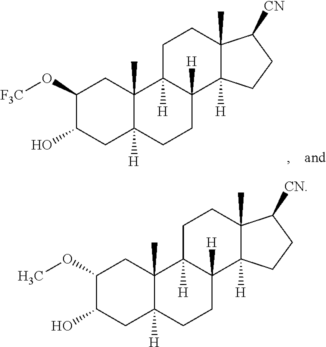

In some embodiments, the compound is selected from:

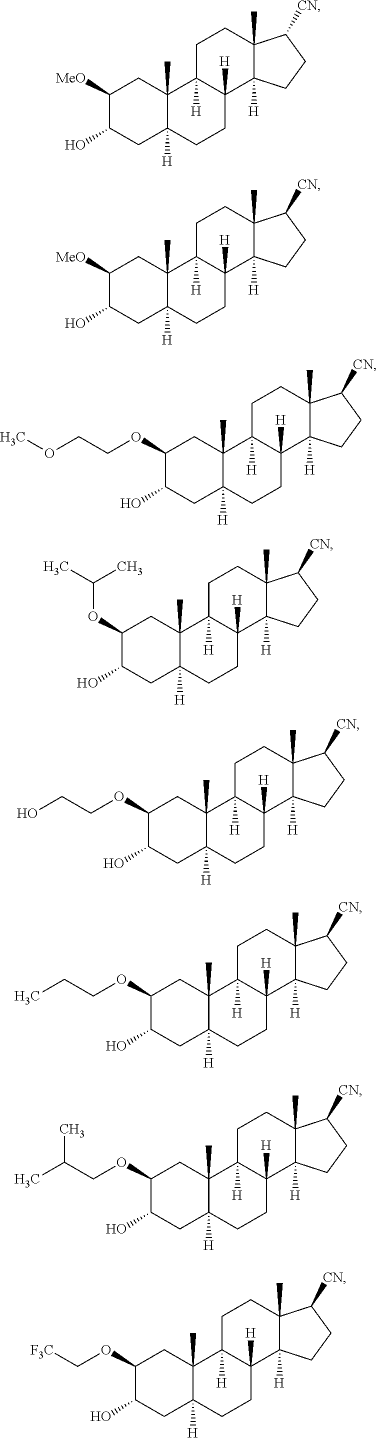

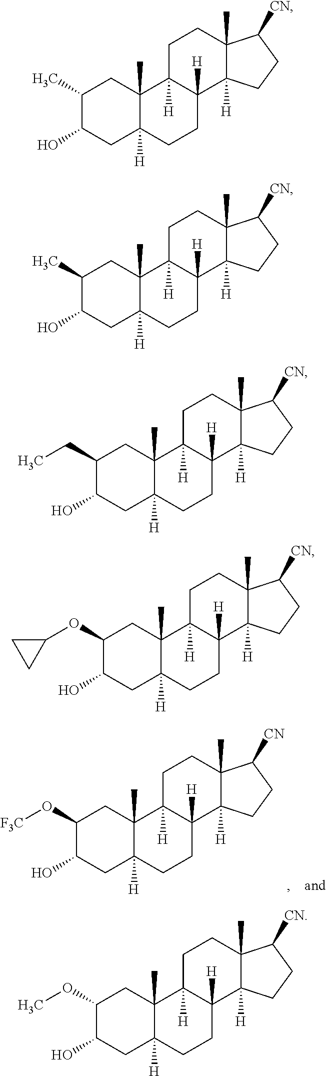

In one aspect, provided is a compound of the Formula (III):

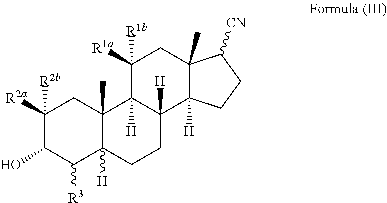

wherein one of R^{1a} and R^{1b} is halo (e.g., chloro, fluoro, bromo, iodo), hydroxy, alkyl, alkoxy, —C(O)R^{a}, —C(O)N(R^{b})(R^{c}), —C(O)OR^{a}, —N(R^{h})(Rⁱ), —OC(O)N(R^{b})(R^{c}), —OC(O)OR^{a}, —OC(O)R^{a}, —S(O)₀₋₂R^{a}, —S(O)₀-2OR^{a}, or —S(O)₀₋₂N(R^{b})(R^{c}), and the other one is hydrogen; or R^{1a} and R^{1b} are taken together with the carbon to which they are attached to form C(═O); one of R^{2a} and R^{2b} is chloro, fluoro, hydroxy, alkyl, alkoxy, —C(O)R^{a}, —C(O)N(R^{b})(R^{c}), —C(O)OR^{a}, —N(R^{f})(R^{g}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}), and the other one is hydrogen; R³ is hydrogen or C₁-C₆ alkyl; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form 3-7-membered (e.g., 5-7-membered) heterocyclic ring; each R^{f} and R^{g} is independently hydrogen or C₁-C₆ alkyl, or R^{f} and R^{g}, taken together with the nitrogen atom to which they are bound to form a 3-7-membered (e.g., 5-7-membered) heterocyclic ring optionally comprising one additional heteroatom selected from nitrogen, oxygen and sulfur; and each R^{h} is unsubstituted C₁-C₄ alkyl; each Rⁱ is hydrogen, substituted methyl or C₂-C₆ alkyl, or R^{h} and Rⁱ, together with the nitrogen atom to which they are attached, form a 3-7-membered (e.g., 5-7-membered) heterocyclic ring.

In some embodiments, R^{1a} is hydrogen and R^{1b} is hydroxy, alkyl, or alkoxy.

In some embodiments, R^{1b} is hydrogen and R^{1a} is hydroxy, alkyl, or alkoxy. In some embodiments, R^{1a} is hydroxy. In some embodiments, R^{1a} is alkoxy. In some embodiments, R^{1a} is methoxy.

In some embodiments, R^{1a} and R^{1b} are taken together with the carbon to which they are attached to form C(═O).

In some embodiments, R^{2a} is hydrogen and R^{2b} is hydroxy, alkyl, or alkoxy. In some embodiments, R^{2b} is hydroxy. In some embodiments, R^{2b} is alkyl. In some embodiments, R^{2b} is methyl. In some embodiments, R^{2b} is alkoxy.

In some embodiments, R^{2b} is hydrogen and R^{2a} is hydroxy, alkyl, or alkoxy. In some embodiments, R^{2a} is hydroxy. In some embodiments, R^{2a} is alkyl. In some embodiments, R^{2a} is methyl. In some embodiments, R^{2a} is alkoxy. In some embodiments, R^{2a} is methoxy. In some embodiments, R^{2a} is ethoxy. In some embodiments, R^{2a} is propoxy. In some embodiments, R^{2a} is —OCH₂CH₂OCH₃. In some embodiments, R^{2a} is —OCH(CH₃)₂.

In some embodiments, R³ is alkyl (e.g., C₁-C₆ alkyl) or alkoxy (e.g., C₁-C₆ alkoxy).

In some embodiments, the compound is a compound of Formula (IIIa):

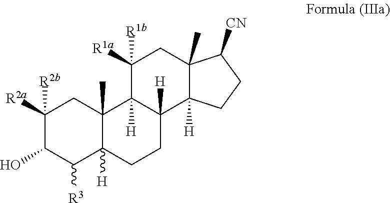

wherein one of R^{1a} and R^{1b} is halo, hydroxy, alkyl, alkoxy, —C(O)R^{a}, —C(O)N(R^{b})(R^{c}), —C(O)OR^{a}, —N(R^{h})(Rⁱ), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}), and the other one is hydrogen; or R^{1a} and R^{1b} are taken together with the carbon to which they are attached to form C(═O); one of R^{2a} and R^{2b} is halo (e.g., chloro, fluoro, bromo, iodo), hydroxy, alkyl, alkoxy, —C(O)R^{a}, —C(O)N(R^{b})(R^{c}), —C(O)OR^{a}, —N(R^{f})(R^{g}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}), and the other one is hydrogen; R³ is hydrogen or C₁-C₆ alkyl; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form 3-7-membered (e.g., 5-7-membered) heterocyclic ring; each R^{f} and R^{g} is independently hydrogen or C₁-C₆ alkyl, or R^{f} and R^{g}, taken together with the nitrogen atom to which they are attached, form a 3-7-membered (e.g., 5-7-membered) heterocyclic ring optionally comprising one additional heteroatom selected from nitrogen, oxygen and sulfur; and each R^{h} is unsubstituted C₁-C₄ alkyl; each Rⁱ is hydrogen, substituted methyl or C₂-C₆ alkyl, or R^{h} and Rⁱ, together with the nitrogen atom to which they are attached, form a 3-7-membered (e.g., 5-7-membered) heterocyclic ring.

In some embodiments, R^{1a} is hydrogen and R^{1b} is hydroxy, alkyl, or alkoxy.

In some embodiments, R^{1b} is hydrogen and R^{1a} is hydroxy, alkyl, or alkoxy. In some embodiments, R^{1a} is hydroxy. In some embodiments, R^{1a} is alkoxy. In some embodiments, R^{1a} is methoxy.

In some embodiments, R^{1a} and R^{1b} are taken together with the carbon to which they are attached to form C(═O).

In some embodiments, R^{2a} is hydrogen and R^{2b} is hydroxy, alkyl, or alkoxy. In some embodiments, R^{2b} is hydroxy. In some embodiments, R^{2b} is alkyl. In some embodiments, R^{2b} is methyl. In some embodiments, R^{2b} is alkoxy.

In some embodiments, R^{2b} is hydrogen and R^{2a} is hydroxy, alkyl, or alkoxy. In some embodiments, R^{2a} is hydroxy. In some embodiments, R^{2a} is alkyl. In some embodiments, R^{2a} is methyl. In some embodiments, R^{2a} is alkoxy. In some embodiments, R^{2a} is methoxy. In some embodiments, R^{2a} is ethoxy. In some embodiments, R^{2a} is propoxy. In some embodiments, R^{2a} is —OCH₂CH₂OCH₃. In some embodiments, R^{2a} is —OCH(CH₃)₂

In some embodiments, R³ is hydrogen. In some embodiments, R³ is C₁-C₆ alkyl.

In some embodiments, the compound is a compound of Formula (IIIb):

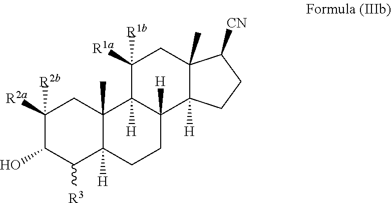

wherein one of R^{1a} and R^{1b} is halo (e.g., chloro, fluoro, bromo, iodo), hydroxy, alkyl, alkoxy, —C(O)R^{a}, —C(O)N(R^{b})(R^{c}), —C(O)OR^{a}, —N(R^{h})(Rⁱ), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}), and the other one is hydrogen; or R^{1a} and R^{1b} are taken together with the carbon to which they are attached to form C(═O); one of R^{2a} and R^{2b} is halo (e.g., chloro, fluoro, bromo, iodo), hydroxy, alkyl, alkoxy, —C(O)R^{a}, —C(O)N(R^{b})(R^{c}), —C(O)OR^{a}, N(R^{f})(R^{g}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}), and the other one is hydrogen; R³ is hydrogen or C₁-C₆ alkyl; each R^{a} is hydrogen or C₁-C₆ alkyl; each R^{b} and R^{c} is independently hydrogen or C₁-C₆ alkyl, or R^{b} and R^{c}, taken together with the nitrogen atom to which they are attached, form 3-7-membered (e.g., 5-7-membered) heterocyclic ring; each R^{f} and R^{g} is independently hydrogen or C₁-C₆ alkyl, or R^{f} and R^{g}, taken together with the nitrogen atom to which they are attached, form a 3-7-membered (e.g., 5-7-membered) heterocyclic ring optionally comprising one additional heteroatom selected from nitrogen, oxygen and sulfur; and each R^{h} is unsubstituted C₁-C₄ alkyl; each Rⁱ is hydrogen, substituted methyl or C₂-C₆ alkyl, or R^{h} and Rⁱ, together with the nitrogen atom to which they are attached, form a 3-7-membered (e.g., 5-7-membered) heterocyclic ring.

In some embodiments, R^{1a} is hydrogen and R^{1b} is hydroxy, alkyl, or alkoxy.

In some embodiments, R^{1b} is hydrogen and R^{1a} is hydroxy, alkyl, or alkoxy. In some embodiments, R^{1a} is hydroxy. In some embodiments, R^{1a} is alkoxy. In some embodiments, R^{1a} is methoxy.

In some embodiments, R^{1a} and R^{1b} are taken together with the carbon to which they are attached to form C(═O).

In some embodiments, R^{2a} is hydrogen and R^{2b} is hydroxy, alkyl, or alkoxy. In some embodiments, R^{2b} is hydroxy. In some embodiments, R^{2b} is alkyl. In some embodiments, R^{2b} is methyl. In some embodiments, R^{2b} is alkoxy.

In some embodiments, R^{2b} is hydrogen and R^{2a} is hydroxy, alkyl, or alkoxy. In some embodiments, R^{2a} is hydroxy. In some embodiments, R^{2a} is alkyl. In some embodiments, R^{2a} is methyl. In some embodiments, R^{2a} is alkoxy. In some embodiments, R^{2a} is methoxy. In some embodiments, R^{2a} is ethoxy. In some embodiments, R^{2a} is propoxy. In some embodiments, R^{2a} is —OCH₂CH₂OCH₃. In some embodiments, R^{2a} is —OCH(CH₃)₂.

In some embodiments, R³ is hydrogen. In some embodiments, R³ is C₁-C₆ alkyl.

In one aspect, provided is a compound selected from:

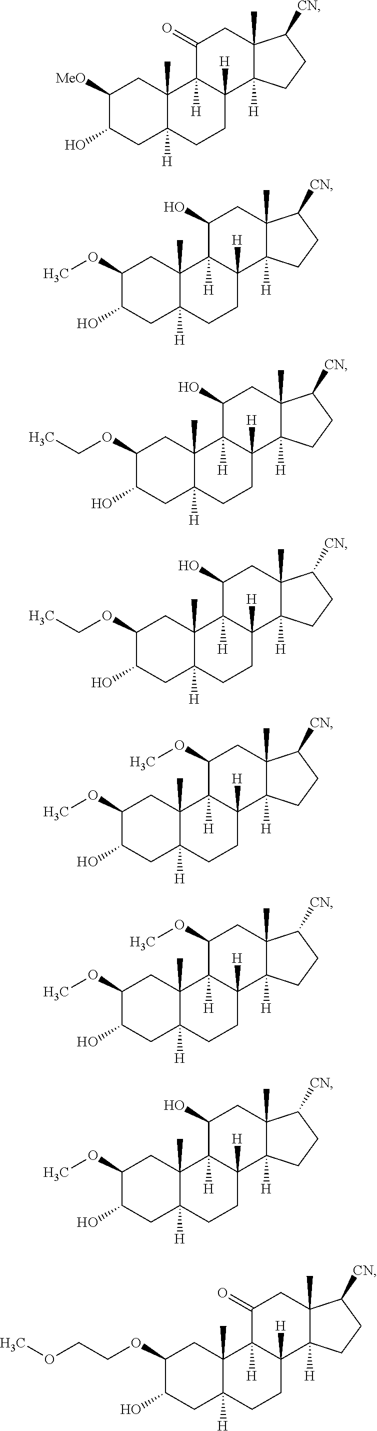

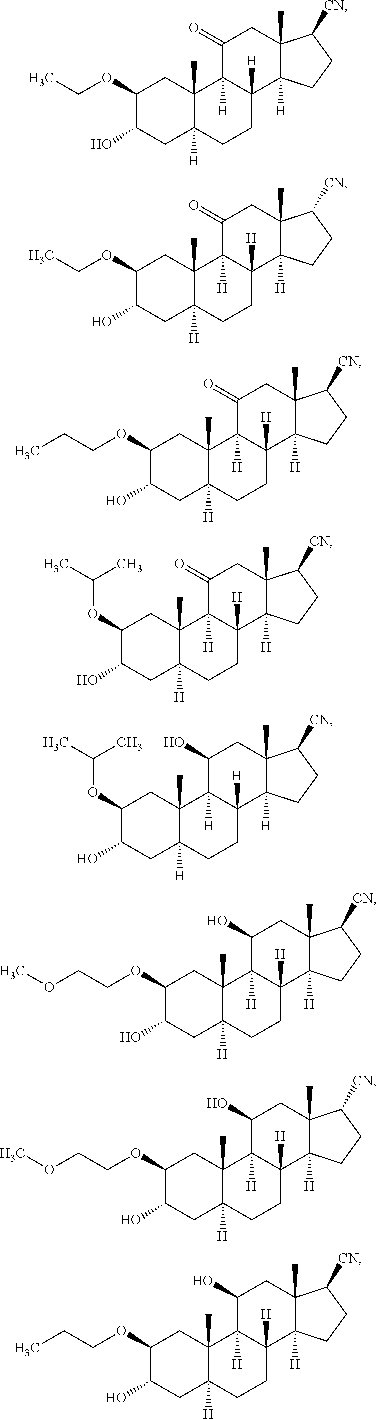

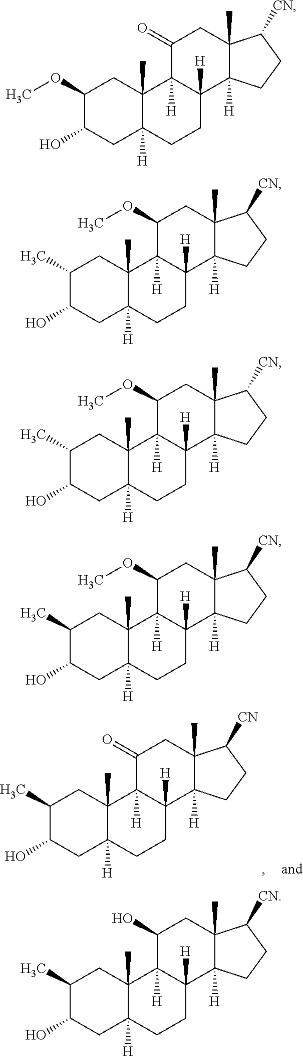

In one aspect, provided is a compound selected from:

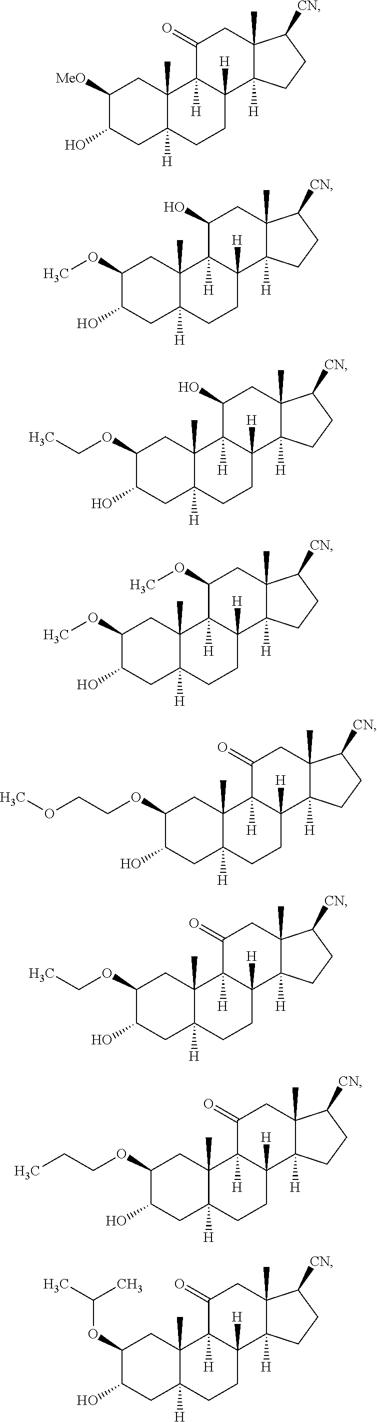

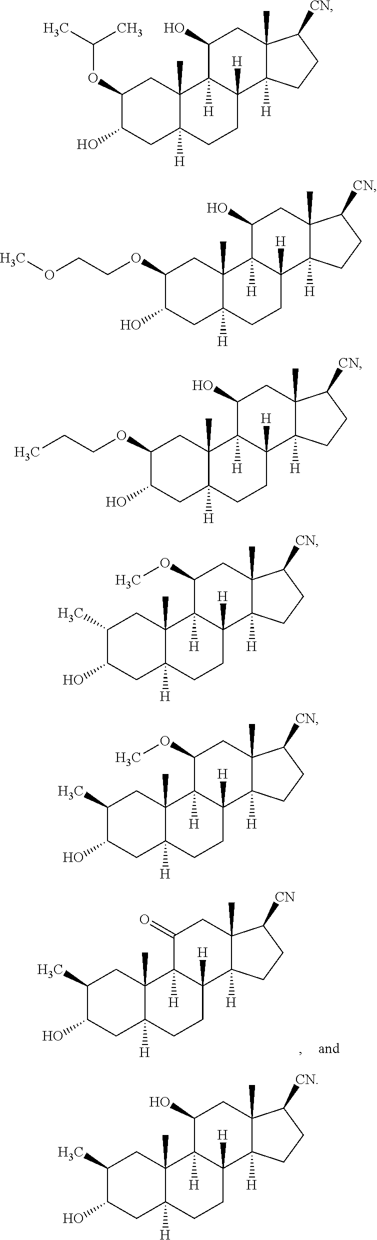

In one aspect, provided is a compound of the Formula (IV):

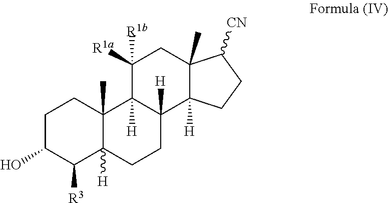

wherein one of R^{1a} and R^{1b} is halo, hydroxy, alkyl, alkoxy, —C(O)R^{a}, —C(O)N(R^{b})(R^{c}), —C(O)OR^{a}, —N(R^{b})(R^{c}), —OC(O)N(R^{b})(R^{c}), —OC(O)OR^{a}, —OC(O)R^{a}, —S(O)₀₋₂R^{a}, —S(O)₀₋₂OR^{a}, or —S(O)₀₋₂N(R^{b})(R^{c}), and the other one is hydrogen; or R^{1a} and R^{1b} are taken together with the carbon to which they are attached to form C(═O); R³ is alkyl or alkoxy; each R^{a} is hydrogen or C₁-C₆ alkyl; each R^{b} and R^{c} is independently hydrogen or C₁-C₆ alkyl, or R^{b} and R^{c}, together with the nitrogen atom to which they are bound to form a 3-7-membered (e.g., 5-7-membered) heterocyclic ring.

In some embodiments, R^{1b} is hydrogen and R^{1a} is hydroxy, alkyl, or alkoxy. In some embodiments, R^{1a} is hydroxy. In some embodiments, R^{1a} is alkyl. In some embodiments, R^{1a} is alkoxy. In some embodiments, R^{1a} is methoxy.

In some embodiments, R^{1a} and R^{1b} are taken together with the carbon to which they are attached to form C(═O).

In some embodiments, R³ is alkyl. In some embodiments, R³ is methyl.

In some embodiments, provided is a compound selected from:

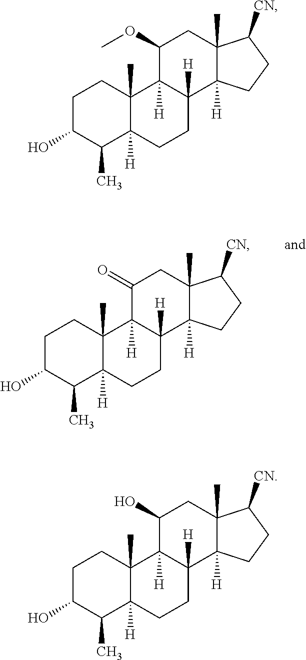

In one aspect, provided is a compound of the Formula (V):

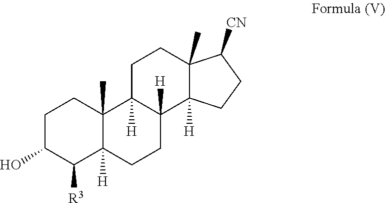

wherein R³ is alkyl or alkoxy.

In some embodiments, R³ is alkyl. In some embodiments, R³ is methyl or ethyl.

In some embodiments, provided is a compound selected from:

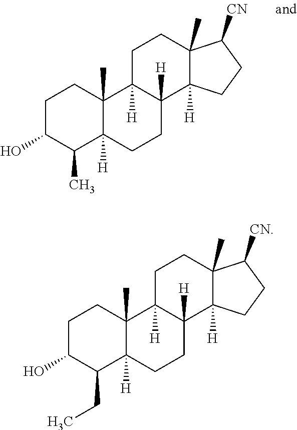

In one aspect, provided is a pharmaceutical composition comprising a compound of Formula (I), (Ia), (Ib), (II), (IIa), (IIb), (III), (IIIa), (IIIb), (IV), or (V) and a pharmaceutically acceptable excipient.

In one aspect, provided is a solvate, isotopic variant, or tautomer of a compound of Formula (I), (Ia), (Ib), (II), (IIa), (IIb), (III), (IIIa), (IIIb), (IV), or (V).

In one aspect, provided herein is a compound having the structure of Formula (VI):

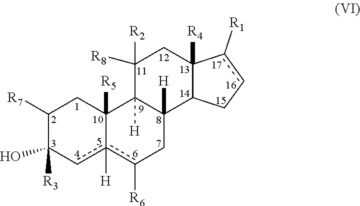

or a pharmaceutically acceptable salt thereof;
wherein:

R₁ is selected from (C₁-C₄ alkyl)-O (e.g., methoxy, ethoxy, propoxy, butoxy), spirooxirane, cyano, ═O, nitro, (C₁-C₄ alkyl)C(O) (e.g., CH₃C(O), CH₃CH₂C(O), CH₃CH₂ CH₂C(O), CH₃CH₂ CH₂ CH₂C(O)), and HO(C₁-C₄ alkyl)C(O) (e.g., HOCH₂C(O), HOCH₂CH₂C(O), HOCH₂CH₂ CH₂C(O), HOCH₂CH₂ CH₂ CH₂C(O)), with R₁ preferably being in the beta position when other than ═O, and/or in one or more preferred embodiments C₁-C₄ alkyl being methyl, R₁ therefore being is selected from methoxy, spirooxirane, cyano, ═O, nitro, CH₃C(O)— and OHCH₂C(O)—;

R₂ is ═O, H, or ORₐ, where Rₐ is selected from hydrogen, optionally substituted C₁-C₄ alkyl, or optionally substituted aryl, with the proviso that when R₂ is ═O, R₈ is not present;

R₃ is hydrogen, optionally substituted C₁-C₄ alkyl, optionally substituted C₂-C₄ alkenyl, optionally substituted C₂-C₄ alkynyl, or optionally substituted aryl;

R₄ is independently selected from hydrogen and unsubstituted C₁-C₄ alkyl;

R₅ is substituted C₁-C₄ alkyl, optionally substituted C₂-C₄ alkenyl, or optionally substituted C₂-C₄ alkynyl (and in particular is alkoxy-substituted methyl, or even more particular is —CH₂—OR_{b}, where R_{b} is C₁-C₄ alkyl, or even still more particularly is —CH₂—OCH₃);

R₆ is hydrogen, optionally substituted C₁-C₄ alkyl, or optionally substituted C₁-C₄ alkoxy;

R₇ is hydrogen, optionally substituted C₁-C₄ alkoxy, or an optionally substituted morpholinyl ring;

R₈, when present, is hydrogen or optionally substituted C₁-C₄ alkyl;
——— denotes an optional, additional C—C bond, resulting in either a C═C bond between C₄-0₅ or C₅-C₆, with the proviso that when present, the R₅-H substituent is not present; and,
——— denotes an optional, additional C—C bond, resulting in a C═C bond between C₁₆-C₁₇, with the proviso that when present, the R₁ is not ═O.

As generally defined above, R₁ is selected from (C₁-C₄ alkyl)-O, spirooxirane, cyano, ═O, nitro, (C₁-C₄ alkyl)C(O), and HO(C₁-C₄ alkyl)C(O). In certain embodiments, R₁ is preferably in the beta position (when other than ═O, or when a C═C is not present between C₁₆-C₁₇). In certain embodiments, R₁ is selected from (C₁-C₄ alkyl)-O (e.g., methoxy, ethoxy, propoxy, butoxy), spirooxirane, cyano, ═O, nitro, (C₁-C₄ alkyl)C(O) (e.g., CH₃C(O), CH₃CH₂C(O), CH₃CH₂ CH₂C(O), CH₃CH₂ CH₂ CH₂C(O)), and HO(C₁-C₄ alkyl)C(O) (e.g., HOCH₂C(O), HOCH₂CH₂C(O), HOCH₂CH₂ CH₂C(O), HOCH₂CH₂ CH₂ CH₂C(O)). In certain embodiments, C₁-C₄ alkyl is methyl, R₁ therefore being is selected from methoxy, spirooxirane, cyano, ═O, nitro, CH₃C(O)— and OHCH₂C(O)—.

As generally defined above, R₂ is ═O, hydrogen, or ORₐ, where Rₐ is selected from hydrogen, optionally substituted C₁-C₄ alkyl, or optionally substituted aryl, with the proviso that when R₂ is ═O, R₈ is not present. In certain embodiments, R₂ is ═O and R₈ is not present. In certain embodiments, R₂ is hydrogen. In certain embodiments, R₂ is ORₐ. In certain embodiments, R₂ is ORₐ and Rₐ is optionally substituted C₁, C₂, C₃, or C₄ alkyl (e.g., methyl, ethyl), optionally substituted benzyl, or C₁, C₂, C₃, or C₄ alkyl substituted with O-aryl, such as O-benzyl. In certain embodiments, R₂ is ORₐ and Rₐ is optionally substituted aryl. In certain embodiments, R₂ is ORₐ and Rₐ is hydrogen.

As generally defined above, R₃ is hydrogen, optionally substituted C₁-C₄ alkyl, optionally substituted C₂-C₄ alkenyl, optionally substituted C₂-C₄ alkynyl, or optionally substituted aryl. In certain embodiments, R₃ is hydrogen. In certain embodiments, R₃ is optionally substituted C₁, C₂, C₃ or C₄ alkyl (e.g., methyl, ethyl, trifluoromethyl, difluoromethyl). In certain embodiments, R₃ is methyl. In certain embodiments, R₃ is trifluoromethyl. In certain embodiments, R₃ is optionally substituted C₂, C₃ or C₄ alkenyl (e.g., optionally substituted allyl). In certain embodiments, R₃ is optionally substituted C₂, C₃, or C₄ alkynyl (e.g., optionally substituted acetylene or optionally substituted propargyl). In certain embodiments, R₃ is optionally substituted aryl (e.g., optionally substituted phenyl, such as phenyl substituted with OH, methyl, or COR_{c}, where R_{c} is optionally substituted C₁-C₂₂ alkyl or optionally substituted C₂-C₂₂ alkenyl, including for example optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl or C₂, C₃, C₄, C₅, C₆, C₇, C_{S}, C₉, C₁₀, C₁₁, C₁₂, C₁₃, C₁₄, C₁₅, C₁₆, C₁₇, C₁₈, C₁₉, C₂₀, C₂₁, or C₂₂ alkenyl).

As generally defined above, R₄ is hydrogen or unsubstituted C₁-C₄ alkyl. In certain embodiments, R₄ is hydrogen. In certain embodiments, R₄ is unsubstituted C₁, C₂, C₃ or C₄ alkyl (e.g., methyl, ethyl, n-propyl, isopropyl, or n-butyl).

As generally defined above, R₅ is substituted C₁-C₄ alkyl, optionally substituted C₂-C₄ alkenyl, or optionally substituted C₂-C₄ alkynyl. In certain embodiments, R₅ is substituted C₁-C₄ alkyl, and in particular is alkoxy-substituted C₁-C₄ alkyl. In other particular embodiments, R₅ is substituted methyl, and more particularly is alkoxy-substituted methyl (or even more particularly is —CH₂—OR_{b}, where R_{b} is C₁-C₄ alkyl, or even still more particularly is —CH₂—OCH₃). In other embodiments, R₅ is optionally substituted C₂-C₄ alkenyl. In other embodiments, R₅ is optionally substituted C₂-C₄ alkynyl.

As generally defined above, R₆ is hydrogen, optionally substituted C₁-C₄ alkyl, or optionally substituted C₁-C₄ alkoxy. In certain embodiments, R₆ is hydrogen. In certain embodiments, R₆ is optionally substituted C₁, C₂, C₃, or C₄ alkyl (e.g., methyl). In certain embodiments, R₆ is optionally substituted C₁, C₂, C₃ or C₄ alkoxy (e.g., methoxy, ethoxy, n-propyloxy, isopropyloxy, or n-butoxy). In certain embodiments, when R₆ is a non-hydrogen group, R₆ is in the alpha (down) position. In certain preferred embodiments, however, when R₆ is a non-hydrogen group, R₆ is in the beta (up) position.

As generally defined above, R₇ is hydrogen, optionally substituted C₁-C₄ alkoxy, or an optionally substituted morpholinyl ring. In certain embodiments, R₇ is hydrogen. In certain embodiments, R₇ is optionally substituted C₁, C₂, C₃ or C₄ alkoxy (e.g., methoxy, ethoxy, n-propyloxy, isopropyloxy, or n-butoxy). In certain embodiments, R₇ is an optionally substituted morpholinyl ring. In certain embodiments, when R₇ is a non-hydrogen group, R₇ is in the alpha (down) position. In certain preferred embodiments, however, when R₇ is a non-hydrogen group, R₇ is in the beta (up) position.

As generally defined above, R₈, when present, is hydrogen or optionally substituted C₁-C₄ alkyl. In certain embodiments, R₈ is hydrogen. In certain embodiments, R₈ is C₁, C₂, C₃ or C₄ optionally substituted alkyl (e.g., methyl). In certain embodiments, when R₈ is optionally substituted C₁-C₄ alkyl, R₈ is in the alpha (down) position. In certain embodiments, when R₈ is optionally substituted C₁-C₄ alkyl, R₈ is in the beta (up) position.

In certain embodiments, R₂ and R₈ are both hydrogen. In certain embodiments, R₂ is ORₐ and R₈ is hydrogen.

As generally defined above, ——— denotes an optional, additional C—C bond, resulting in either a C═C bond between C₄-0₅ or Cₛ-C₆, with the proviso that when present, the C₅—H substituent is not present. In certain embodiments, the additional C—C bond is absent, and the hydrogen at C₅ is in the alpha or beta position. In certain embodiments, the additional C—C bond is absent, and the hydrogen at C₅ is in the alpha (down) position. In certain embodiments, the additional C—C bond is absent, and the hydrogen at C₅ is in the beta (up) position. In certain embodiments, ——— denotes an additional C—C bond, resulting in a C═C bond between C₄-0₅. In certain embodiments, ——— denotes an additional C—C bond, resulting in a C═C bond between C₅-C₆.

As generally defined above, ——— denotes an optional, additional C—C bond, resulting in a C═C bond, between C₁₆-C₁₇, with the proviso that when present, the R₁ is other than ═O. In certain embodiments, the additional C—C bond is absent (i.e., there is not C═C bond), and therefore R₁ may be in the alpha or beta position. In certain embodiments, the additional C—C bond is absent, and the R₁ is in the alpha (down) position. In certain embodiments, the additional C—C bond is absent, and the R₁ is in the beta (up) position.

It is to be noted that the present disclosure contemplates and is intended to encompass all of the various combinations and permutations (i.e., combinations of substituent options, locations and stereochemical configurations) possible here.

For example, in various embodiments, compounds of the present disclosure may be selected from among those encompassed by the structure of Formula (VI), wherein R₂ is ═O; alternatively, R₂ may be hydrogen and R₈ is hydrogen (e.g., C₁₁ thus having two hydrogen atoms bound thereto as substituents). In certain embodiments, R₂ may be ORₐ, wherein Rₐ is methyl, optionally substituted benzyl, or C₁-C₄ alkyl substituted with O-aryl, such as O-benzyl. In certain embodiments, R₃ may be hydrogen, methyl, trifluoromethyl, or substituted aryl (e.g., substituted phenyl, which in turn may be optionally substituted such as, for example, with OH, methyl, or COR_{c}, where R_{c}═C₁-C₄ alkyl); further, when R₃ is something other than hydrogen, R₃ is preferably in the β-position. In certain embodiments, each of R₄ and R₆ are independently selected from hydrogen and methyl, R₅ being in the β-configuration and R₆ optionally being in the a-configuration or β-configuration (e.g., when R₆ is methyl), which the β-configuration being preferred. In certain embodiments, R₇ is selected from hydrogen, methoxy, ethoxy, and an optionally substituted morpholinyl ring; further, when R₇ is something other than hydrogen, R₇ is preferably in the β-position. In certain embodiments, R₈, when present, is selected from hydrogen or optionally substituted C₁-C₄ alkyl. In certain embodiments, R₈ is methyl (e.g., methyl in the alpha-configuration).

In certain embodiments, the C₅—H is in the alpha configuration and the R₅ is, for example, a substituted methyl group (e.g., alkoxy-substituted methyl, or in particular a methoxy-substituted methyl) in the beta configuration. In certain embodiments, the C5—H is in the beta configuration and R₅ is, for example, a substituted methyl (e.g., a methoxy-substituted methyl) group in the beta configuration. In certain embodiments, R₆ is hydrogen. In certain embodiments, R₄ is methyl. In certain embodiments, R₂ is ═O or methoxy.

Accordingly, as noted, the steroid of Formula (VI) may encompass a number of various structures in accordance with the present disclosure.

In certain embodiments, wherein R₁ is as defined above, R₃ is in the beta position, R₄ is methyl, R₅ is substituted methyl in the beta position, and R₆ is hydrogen, provided is a compound of Formula (VI-a):

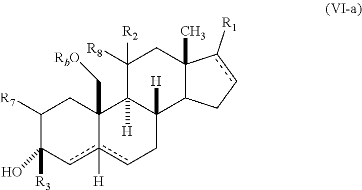

or a pharmaceutically acceptable salt thereof, wherein ———, R₂, R₃, R₇ and R₈ are as defined herein, and further wherein R_{b} is optionally substituted C₁-C₄ alkyl. In certain embodiments, each instance of ——— between C₅-C₆ and C₆-C₇ is absent and C₅—H is in the alpha position. In certain embodiments, each instance of ——— between C₅-C₆ and C₆-C₇ is absent and C₅—H is in the beta position. In certain embodiments, each instance of ——— between C₁₆-C₁₇ is absent and R₁ is in the beta position.

In certain embodiments of Formula (VI), wherein R₂ is ═O and R₈ is absent, provided is a compound of Formula (VI-b):

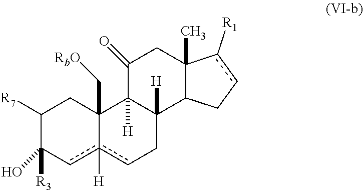

or a pharmaceutically acceptable salt thereof, wherein ———, R₃ and R₇ are as defined herein, and further wherein R_{b} is optionally substituted C₁-C₄ alkyl. In certain embodiments, each instance of ——— between C₅-C₆ and C₆-C₇ is absent and C₅—H is in the alpha position. In certain embodiments, each instance of ——— between C₅-C₆ and C₆-C₇ is absent and C₅—H is in the beta position. In certain embodiments, each instance of ——— between C₁₆-C₁₇ is absent and R₁ is in the beta position.

In certain embodiments of Formula (VI), wherein R₂ and R₈ are hydrogen, provided is a compound of Formula (VI-c):

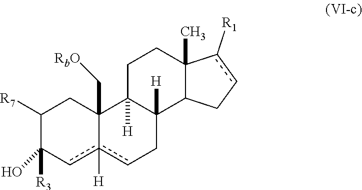

or a pharmaceutically acceptable salt thereof, wherein ———, R₂, R₃, and R₇ are as defined herein, and further wherein R_{b} is optionally substituted C₁-C₄ alkyl. In certain embodiments, each instance of ——— between C₅-C₆ and C₆-C₇ is absent and C₅—H is in the alpha position. In certain embodiments, each instance of ——— between C₅-C₆ and C₆-C₇ is absent and C₅—H is in the beta position. In certain embodiments, each instance of ——— between C₁₆-C₁₇ is absent and R₁ is in the beta position.

In certain embodiments of Formula (VI), wherein R₂ is ORₐ and R₈ is hydrogen, provided is a compound of Formula (VI-d):

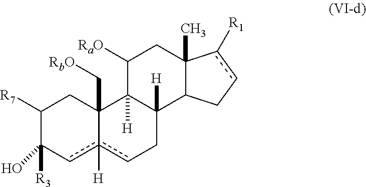

or a pharmaceutically acceptable salt thereof, wherein ———, R₃, R₇, and Rₐ are as defined herein, and further wherein R_{b} is optionally substituted C₁-C₄ alkyl. In certain embodiments, each instance of ——— between C₅-C₆ and C₆-C₇ is absent and C₅—H is in the alpha position. In certain embodiments, each instance of ——— between C₅-C₆ and C₆-C₇ is absent and C₅—H is in the beta position. In certain embodiments, each instance of ——— between C₁₆-C₁₇ is absent and R₁ is in the beta position.

In certain embodiments of Formula (VI), wherein R₇ is hydrogen, provided is a compound of Formula (VI-e):

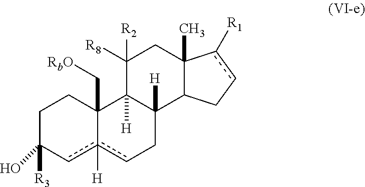

or a pharmaceutically acceptable salt thereof, wherein ———, R₂, R₃, and R₈ are as defined herein, and further wherein R_{b} is optionally substituted C₁-C₄ alkyl. In certain embodiments, each instance of ——— between C₅-C₆ and C₆-C₇ is absent and C₅—H is in the alpha position. In certain embodiments, each instance of ——— between C₅-C₆ and C₆-C₇ is absent and C₅—H is in the beta position. In certain embodiments, each instance of ——— between C₁₆-C₁₇ is absent and R₁ is in the beta position.

In certain embodiments of Formula (VI), wherein each instance of ——— is absent and C₅—H is in the alpha position, provided is a compound of Formula (VI-f):

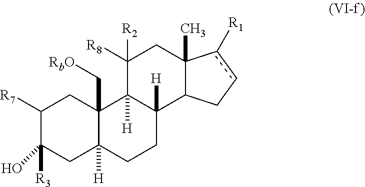

or a pharmaceutically acceptable salt thereof, wherein R₂, R₃, R₇ and R₈ are as defined herein, and further wherein R_{b} is optionally substituted C₁-C₄ alkyl. In certain embodiments, each instance of ——— between C₁₆-C₁₇ is absent and R₁ is in the beta position.

In certain embodiments of Formula (VI), wherein R₇ is hydrogen, provided is a compound of Formula (VI-g):

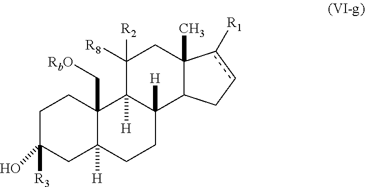

or a pharmaceutically acceptable salt thereof, wherein R₂, R₃, and R₈ are as defined herein, and further wherein R_{b} is optionally substituted C₁-C₄ alkyl. In certain embodiments, each instance of ——— between C₁₆-C₁₇ is absent and R₁ is in the beta position.

In certain embodiments of Formula (VI), wherein R₂ is ═O, provided is a compound of Formula (VI-h):

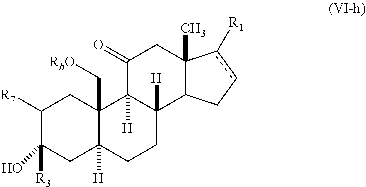

or a pharmaceutically acceptable salt thereof, wherein R₃ and R₇ are as defined herein, and further wherein R_{b} is optionally substituted C₁-C₄ alkyl. In certain embodiments, each instance of ——— between C₁₆-C₁₇ is absent and R₁ is in the beta position.

In certain embodiments of Formula (VI), wherein R₂ is ORₐ, provided is a compound of Formula (VI-i):

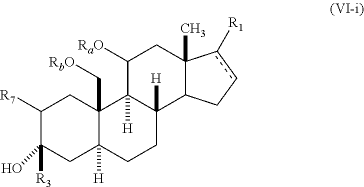

or a pharmaceutically acceptable salt thereof, wherein Rₐ, R₃, and R₇ are as defined herein, and further wherein R_{b} is optionally substituted C₁-C₄ alkyl. In certain embodiments, each instance of ——— between C₁₆-C₁₇ is absent and R₁ is in the beta position.
In certain embodiments of Formula (VI), wherein ——— represents an additional C—C bond, resulting in a C═C bond between C₄-0₅ provided is a compound of Formula (VI-j):

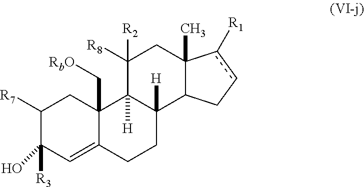

or a pharmaceutically acceptable salt thereof, wherein R₃, R₂, R₇ and R₈ are as defined herein, and further wherein R_{b} is optionally substituted C₁-C₄ alkyl. In certain embodiments, each instance of ——— between C₁₆-C₁₇ is absent and R₁ is in the beta position. In certain embodiments of Formula (VI), wherein ——— represents an additional C—C bond, resulting in a C═C bond between C₅-C₆ provided is a compound of Formula (VI-k):

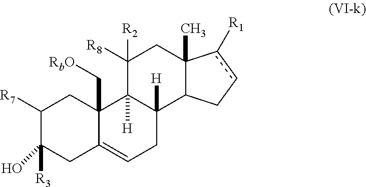

or a pharmaceutically acceptable salt thereof, wherein R₃, R₂, R₇ and R₈ are as defined herein, and further wherein R_{b} is optionally substituted C₁-C₄ alkyl. In certain embodiments, each instance of ——— between C₁₆-C₁₇ is absent and R₁ is in the beta position.
It is to be noted that, in one or more of the preferred embodiments detailed above, R₁ may, in particular, be selected from methoxy (or more generally lower alkoxy, e.g., —O-(C₁-C₄)), or alternatively selected from CH₃C(O)— or HOCH₂C(O)— (or more generally substituted or unsubstituted lower alkyl-carbonyl, e.g., (C₁-C₄)C(O)—, wherein one or more of the carbon atoms is optionally substituted, such as for example by a hydroxyl group). Alternatively, R₁ may be selected from nitro or cyano, with an optional C═C being present between C₁₆-C₁₇. In yet another alternative embodiment, C₁₇ may be a carbonyl carbon (i.e., R₁ is ═O), or it may be part of a oxirane ring fused with the D-ring (i.e., R₁ being a spirooxirane substituent, wherein C₁₇ is the carbon atom common to both rings).

Exemplary compounds of Formula (VI) include, but are not limited to, the following:

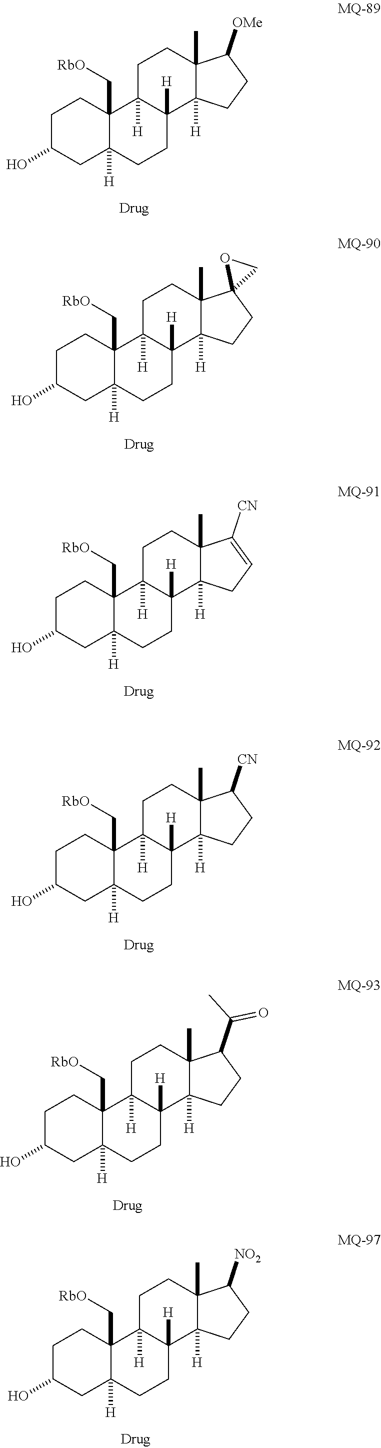

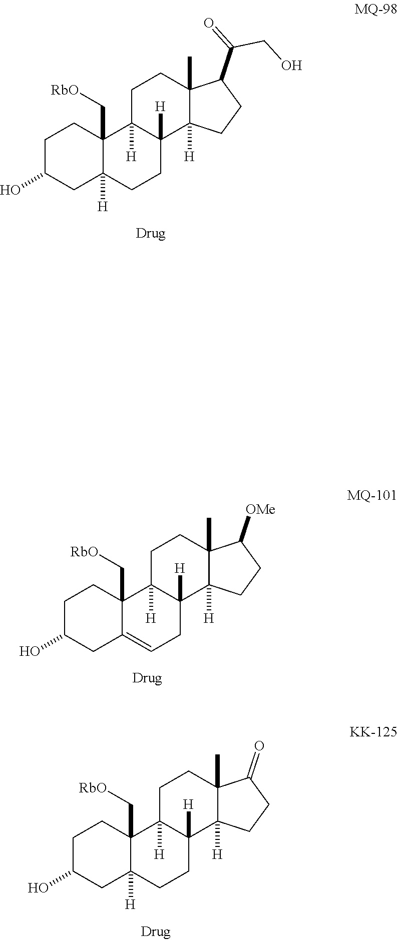

and pharmaceutically acceptable salts thereof, wherein in one preferred embodiment R_{b} is CH₃. In certain embodiments, the steroid of Formula (VI) is selected from the group consisting of:

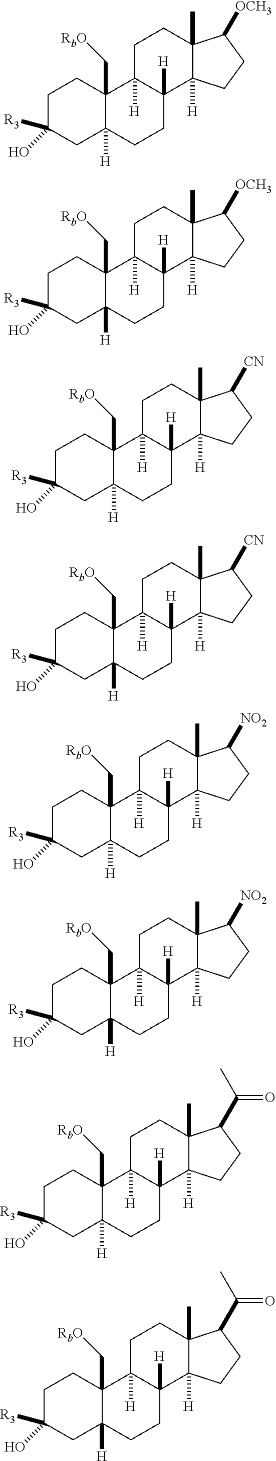

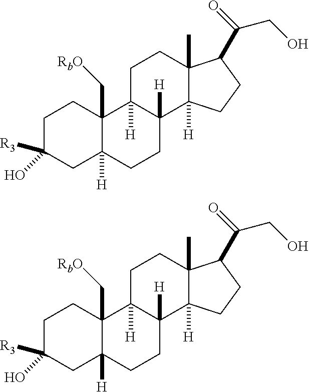

and pharmaceutically acceptable salts thereof, wherein R₃ is as defined above, and in one particular embodiment is hydrogen, and further wherein in this or another preferred embodiment R_{b} is CH₃.

In certain embodiments, the steroid of Formula (VI) is selected from the group consisting of:

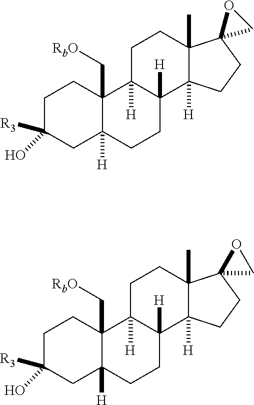

and pharmaceutically acceptable salts thereof, wherein R₃ is as defined above, and in one particular embodiment is hydrogen, and further wherein in this or another preferred embodiment R_{b} is CH₃.

In certain embodiments, the steroid of Formula (VI) is selected from the group consisting of:

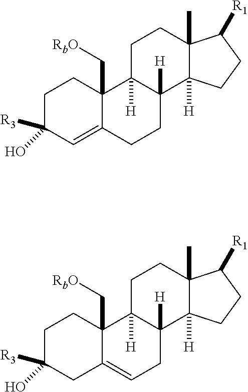

and a pharmaceutically acceptable salt thereof, wherein R₃ and/or R₁ are as defined above, and in one particular embodiment R₃ is hydrogen and R₁ is methoxy, and further wherein in these or other preferred embodiments R_{b} is CH₃.

In certain embodiments, the steroid of Formula (VI) is selected from the group consisting of:

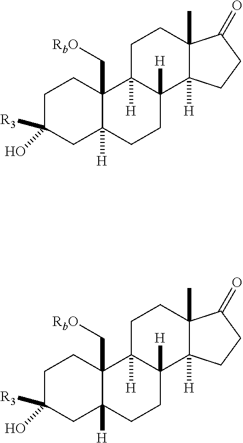

and a pharmaceutically acceptable salt thereof, wherein R₃ is as defined above, and in one particular embodiment is hydrogen, and further wherein in this or another preferred embodiment R_{b} is CH₃.

In certain embodiments, the steroid of Formula (VI) is selected from the group consisting of:

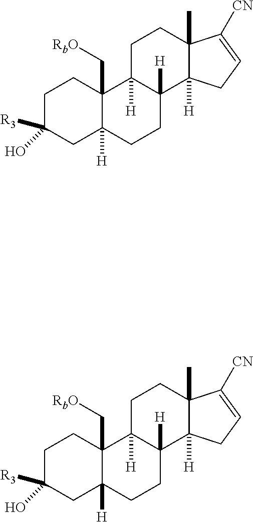

and a pharmaceutically acceptable salt thereof, wherein R₃ is as defined above, and in one particular embodiment is hydrogen, and further wherein in this or another preferred embodiment R_{b} is CH₃.
[1] In an aspect, provided herein is a compound of the Formula (VII):

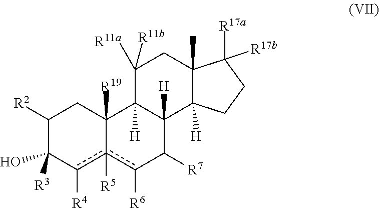

or a pharmaceutically acceptable salt thereof, wherein: each of R², R⁴, R⁶, R⁷, R^{11a} and R^{11b} is independently hydrogen, halogen, cyano, nitro, alkyl, alkenyl, alkynyl, carbocyclyl, heterocyclyl, aryl, heteroaryl, —OR^{A1}, —SR^{A1}, —N(R^{A1})₂, —NHC(═O)R^{A1}, —S(═O)R^{A2}, —SO₂R^{A2}, or —S(═O)₂OR^{A1}, wherein each instance of R^{A1} is independently hydrogen, alkyl, alkenyl, alkynyl, carbocyclyl, heterocyclyl, aryl, heteroaryl, an oxygen protecting group when attached to an oxygen atom, a sulfur protecting group when attached to a sulfur atom, a nitrogen protecting group when attached to a nitrogen atom, or two R^{A1} groups are joined to form an heterocyclic or heteroaryl ring; and R^{A2} is alkyl, alkenyl, alkynyl, carbocyclyl, heterocyclyl, aryl, or heteroaryl; or R^{11a} and R^{11b} together with the carbon atom to which they are attached form a carbocyclyl, heterocyclyl, or —C(═O)—; R³ is hydrogen, alkyl, alkenyl, carbocyclyl, heterocyclyl, aryl, or heteroaryl; each of R^{17a} and R^{17b} is independently hydrogen, halogen, cyano, nitro, alkyl, alkenyl, alkynyl, carbocyclyl, heterocyclyl, aryl, heteroaryl, —OR^{A1}, —SR^{A1}, —N(R^{A1})₂,—NHC(═O)R^{A1}—S(═O)R^{A2}, —SO₂R^{A2}, or —S(═O)₂OR^{A1}, wherein at least one of R^{17a} and R^{17b} is not hydrogen; R¹⁹ is hydrogen or alkyl (e.g., unsubstituted alkyl or substituted alkyl (e.g., —C(R^{C})₂OR^{A1}, wherein R^{C} is hydrogen or alkyl)); R⁵ is absent or hydrogen; and [symbol] represents a single or double bond, wherein when one [symbol] is a double bond, the other [symbol] is a single bond and R⁵ is absent.

In some embodiments, R¹⁹ is hydrogen or alkyl. In some embodiments, R¹⁹ is unsubstituted alkyl. In some embodiments, R¹⁹ is substituted alkyl. In some embodiments, R¹⁹ is —CH₂OH, —CH₂OCH₃, —CH₂OCH₂CH₃, or —CH₂OCH(CH₃)₂.

In some embodiments, R² is hydrogen, halogen, alkyl, carbocyclyl, heterocyclyl, aryl, heteroaryl, —OR^{A1}, —SR^{A1}, or —N(R^{A1})₂. In some embodiments, R² is hydrogen, halogen, alkyl, or —OR^{A1}. In some embodiments, R² is hydrogen.

In some embodiments, R³ is alkyl, alkenyl, carbocyclyl, heterocyclyl, aryl, or heteroaryl.

In some embodiments, R³ is alkyl (e.g., substituted or unsubstituted alkyl). In some embodiments, R³ is methyl and ethyl (e.g., substituted or unsubstituted alkyl).

In some embodiments, R⁴ is hydrogen, halogen, alkyl, carbocyclyl, heterocyclyl, aryl, heteroaryl, —OR^{A1}, —SR^{A1}, or —N(R^{A1})₂. In some embodiments, R⁴ is hydrogen, halogen, alkyl, or —OR^{A1}. In some embodiments, R⁴ is hydrogen.

In some embodiments, [symbol] represents a single bond and R⁵ is hydrogen. In some embodiments, R⁵ is absent, and [symbol] represents a single or double bond, wherein when one [symbol] is a double bond, the other [symbol] is a single bond.

In some embodiments, R⁶ is hydrogen, halogen, alkyl, carbocyclyl, heterocyclyl, aryl, heteroaryl, —OR^{A1}, —SR^{A1}, or —N(R^{A1})₂. In some embodiments, R⁶ is hydrogen, halogen, alkyl, or —OR^{A1}. In some embodiments, R⁶ is hydrogen.

In some embodiments, R⁷ is hydrogen, halogen, alkyl, carbocyclyl, heterocyclyl, aryl, heteroaryl, —OR^{A1}, —SR^{A1}, or —N(R^{A1})₂. In some embodiments, R⁷ is hydrogen, halogen, alkyl, or —OR^{A1}. In some embodiments, R⁷ is hydrogen.

In some embodiments, R^{11a} is hydrogen, halogen, alkyl, carbocyclyl, heterocyclyl, aryl, heteroaryl, —OR^{A1}, —SR^{A1}, —N(R^{A1})₂, or R^{11a} and R^{11b} together with the carbon atom to which they are attached form C(═O). In some embodiments, R^{11a} is hydrogen, halogen, alkyl, or —OR^{A1}. In some embodiments, R^{11a} and R^{11b} together with the carbon atom to which they are attached form —C(═O)—. In some embodiments, R^{11a} and R^{11b} are hydrogen.

In some embodiments, each of R², R⁴, R⁶, R⁷, R^{11a}, and R^{11b} is independently hydrogen, halogen, alkyl, carbocyclyl, heterocyclyl, aryl, heteroaryl, —OR^{A1}, —SR^{A1}, or —N(R^{A1})₂. In some embodiments, each of R², R⁴, R⁶, R⁷, R^{11a} and R^{11b} is independently hydrogen, halogen, alkyl, or —OR^{A1}. In some embodiments, R², R⁴, R⁶, R⁷, R^{11a}, and R^{11b} are hydrogen.

In some embodiments, each of R^{17a} and R^{17b} is independently hydrogen, halogen, cyano, nitro, alkyl, alkenyl, alkynyl, carbocyclyl, heterocyclyl, —SR^{A1}, —N(R^{A1})₂,—NHC(═O)R^{A1}—S(═O)R^{A2}, —SO₂R^{A2}, or —S(═O)₂OR^{A1}. In some embodiments, each of R^{17a} and R^{17b} is independently hydrogen, halogen, nitro, alkyl, alkenyl, alkynyl, carbocyclyl, heterocyclyl, —SR^{A1}, —N(R^{A1})₂,—NHC(═O)R^{A1}—S(═O)R^{A2}, —SO₂R^{A2}, or —S(═O)₂OR^{A1}.

In some embodiments, R^{17a} is halogen, cyano, nitro, alkyl, carbocyclyl, heterocyclyl, —OR^{A1}, —SR^{A1}, —N(R^{A1})₂, —NHC(═O)R^{A1}, —S(═O)R^{A2}, or —SO₂R^{A2}. In some embodiments, R^{17a} is halogen, cyano, nitro, alkyl,—OR^{A1}, —SR^{A1}, or —N(R^{A1})₂. In some embodiments, R^{17a} is halogen, nitro, alkyl, carbocyclyl, heterocyclyl, —OR^{A1}, —SR^{A1}, —N(R^{A1})₂, —NHC(═O)R^{A1}, —S(═O)R^{A2}, or —SO₂R^{A2}, wherein at least one of R^{17a} and R^{17b} is not hydrogen.

In some embodiments, the compound is:

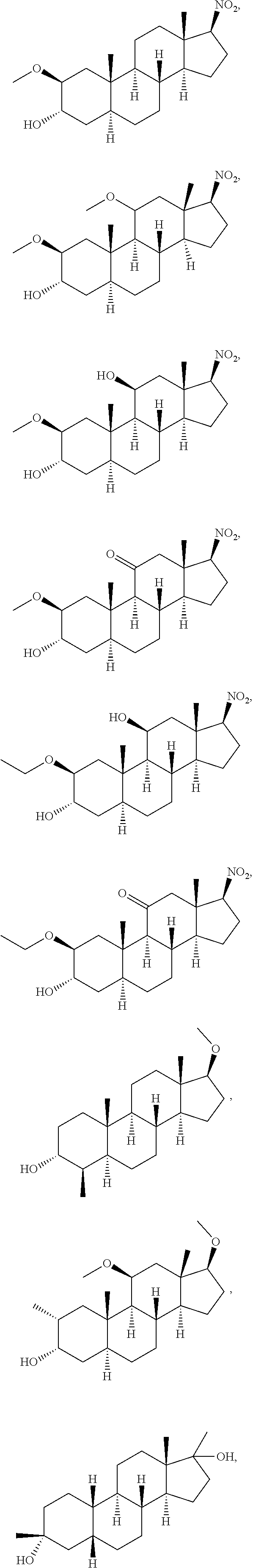

or a pharmaceutically acceptable salt thereof.

### Methods of Use and Treatment

The compounds described herein can be used, for example, to treat an injury or disorder in a subject who has been exposed to a chemical warfare agent. In some embodiments, the chemical warfare agent is a nerve agent or toxin. In some embodiments, the injury or disorder is a seizure.

In an aspect, provided is a method of alleviating or preventing seizure activity in a subject, comprising administering to the subject in need of such treatment an effective amount of a compound of the present invention. In some embodiments, the method alleviates or prevents epileptogenesis.

In some embodiments, such compounds are envisioned to be useful as therapeutic agents for treating a CNS-related disorder (e.g., sleep disorder, a mood disorder such as depression, a schizophrenia spectrum disorder, a convulsive disorder, epileptogenesis, a disorder of memory and/or cognition, a movement disorder, a personality disorder, autism spectrum disorder, pain, traumatic brain injury, a vascular disease, a substance abuse disorder and/or withdrawal syndrome, or tinnitus) in a subject in need (e.g., a subject with Rett syndrome, Fragile X syndrome, or Angelman syndrome). Exemplary CNS conditions related to GABA-modulation include, but are not limited to, sleep disorders [e.g., insomnia], mood disorders [e.g., depression, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD))], schizophrenia spectrum disorders [e.g., schizophrenia, schizoaffective disorder], convulsive disorders [e.g., epilepsy (e.g., status epilepticus (SE)), seizures], disorders of memory and/or cognition [e.g., attention disorders (e.g., attention deficit hyperactivity disorder (ADHD)), dementia (e.g., Alzheimer's type dementia, Lewis body type dementia, vascular type dementia], movement disorders [e.g., Huntington's disease, Parkinson's disease], personality disorders [e.g., anti-social personality disorder, obsessive compulsive personality disorder], autism spectrum disorders (ASD) [e.g., autism, monogenetic causes of autism such as synaptophathy's, e.g., Rett syndrome, Fragile X syndrome, Angelman syndrome], pain [e.g., neuropathic pain, injury related pain syndromes, acute pain, chronic pain], traumatic brain injury (TBI), vascular diseases [e.g., stroke, ischemia, vascular malformations], substance abuse disorders and/or withdrawal syndromes [e.g., addition to opiates, cocaine, and/or alcohol], and tinnitus.

In yet another aspect, provided is a combination of a compound of the present invention and another pharmacologically active agent. The compounds provided herein can be administered as the sole active agent or they can be administered in combination with other agents. Administration in combination can proceed by any technique apparent to those of skill in the art including, for example, separate, sequential, concurrent and alternating administration.

In another aspect, provided is a method of treating or preventing brain excitability in a subject susceptible to or afflicted with a condition associated with brain excitability, comprising administering to the subject an effective amount of a compound of the present invention to the subject.

In yet another aspect, provided is a method of treating or preventing stress or anxiety in a subject, comprising administering to the subject in need of such treatment an effective amount of a compound of the present invention, or a composition thereof.

In yet another aspect, provided is a method of alleviating or preventing insomnia in a subject, comprising administering to the subject in need of such treatment an effective amount of a compound of the present invention, or a composition thereof.

In yet another aspect, provided is a method of inducing sleep and maintaining substantially the level of REM sleep that is found in normal sleep, wherein substantial rebound insomnia is not induced, comprising administering an effective amount of a compound of the present invention.

In yet another aspect, provided is a method of alleviating or preventing PMS or PND in a subject, comprising administering to the subject in need of such treatment an effective amount of a compound of the present invention.

In yet another aspect, provided is a method of treating or preventing mood disorders in a subject, comprising administering to the subject in need of such treatment an effective amount of a compound of the present invention. In certain embodiments the mood disorder is depression.

In yet another aspect, provided is a method of cognition enhancement or treating memory disorder by administering to the subject a therapeutically effective amount of a compound of the present invention. In certain embodiments, the disorder is Alzheimer's disease. In certain embodiments, the disorder is Rett syndrome.

In yet another aspect, provided is a method of treating attention disorders by administering to the subject a therapeutically effective amount of a compound of the present invention. In certain embodiments, the attention disorder is ADHD.

In certain embodiments, the compound is administered to the subject chronically. In certain embodiments, the compound is administered to the subject orally, subcutaneously, intramuscularly, or intravenously.

### Seizure

The compounds and methods described herein can be used, for example, to treat an injury or disorder in a subject who has been exposed to a chemical warfare agent. In some embodiments, the chemical warfare agent is a nerve agent or toxin. In some embodiments, the injury or disorder is a seizure.

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

In some embodiments, the seizure is induced by a warfare agent (e.g., a chemical warfare agent). For example, in some embodiments, the seizure is induced by a nerve agent or toxin.

### Epilepsy

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Epileptogenesis

The compounds and methods described herein can be used to treat or prevent epileptogenesis. Epileptogenesis is a gradual process by which a normal brain develops epilepsy (a chronic condition in which seizures occur). Epileptogenesis results from neuronal damage precipitated by the initial insult (e.g., status epilepticus).

### Anxiety Disorders

Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of Phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Neurodegenerative Diseases and Disorders

The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

### Pharmaceutical Compositions

In one aspect, the invention provides a pharmaceutical composition comprising a compound of the present invention (also referred to as the “active ingredient”) and a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 20 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 5 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

In certain embodiments, the pharmaceutical composition further comprises a cyclodextrin derivative. The most common cyclodextrins are α-, β- and γ-cyclodextrins consisting of 6, 7 and 8 α-1 ,4linked glucose units, respectively, optionally comprising one or more substituents on the linked sugar moieties, which include, but are not limited to, substituted or unsubstituted methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol®. See, e.g., U.S. Pat. No. 5,376,645. In certain embodiments, the composition comprises hexapropyl-β-cyclodextrin. In a more particular embodiment, the composition comprises hexapropyl-β-cyclodextrin (10-50% in water).

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for treating a subject having an injury resulting from exposure to a warfare agent, the method comprising administering to the subject a compound described herein.

2. A method of treating an injury in a subject who has been exposed to a chemical warfare agent, the method comprising administering to the subject a compound described herein.

3. A method of treating a subject, the method comprising:
identifying a subject that has been exposed to a chemical warfare agent such as a nerve agent or toxin; and administering to the subject a compound described herein.

4. The method of claim 1, wherein the injury is a seizure.

5. The method of claim 4, wherein the injury is a myoclonic seizure.

6. The method of claim 1, wherein the injury is status epilepticus.

7. The method of claim 1, wherein the administration is within 1 week; 6, 5, 4, 3, 2, 1 day; 24, 22, 20, 18, 16, 14, 12, 10, 8, 7, 6, 5, 4, 3, 2, 1 hour, 45, 30, 20, 10, or 5 minutes of exposure to the chemical warfare agent.

8. The method of one of claims 1-3, wherein the compound is administered parenterally.

9. The method of claim 1, wherein the compound is administered by intravenous administration.

10. The method of claim 1, wherein the subject is a human.

11. The method of claim 1, wherein the chemical warfare agent is a nerve agent or toxin.

12. The method of claim 11, wherein the chemical warfare agent is a nerve agent.

13. The method of claim 12, wherein the nerve agent is a phosphorus-containing organic chemical.

14. The method of claim 12, wherein the nerve agent is a G agent.

15. The method of claim 12, wherein the nerve agent is a V agent.

16. The method of claim 11, wherein the toxin is abrin, ricin, or saxitoxin.

17. The method of claim 1, wherein the compound is a compound as described herein.

18. A method for treating disorders related to GABA function in a subject in need thereof, the method comprising administering to the subject a therapeutically effective amount of a compound, a pharmaceutically acceptable salt thereof, or pharmaceutical composition of a compound of claim 1.

19. A method for treating a CNS-related disorder in a subject in need thereof, comprising administering to the subject an effective amount of a compound of claim 1, or a pharmaceutically acceptable salt thereof.

20. The method of claim 19, wherein the CNS-related disorder is a sleep disorder, a mood disorder such as depression, a schizophrenia spectrum disorder, a convulsive disorder, epileptogenesis, a disorder of memory and/or cognition, a movement disorder, a personality disorder, autism spectrum disorder, pain, traumatic brain injury, a vascular disease, a substance abuse disorder and/or withdrawal syndrome, or tinnitus.

21. A kit comprising a solid composition comprising a compound of claim 1 and a sterile diluent.
